(19) United States
(12) Patent Application Publication
(10) Pub. No.: US 2023/0173096 A1
(43) Pub. Date: Jun. 8, 2023

(54) TARGETED ANTIVIRAL DRUGS

(71) Applicant: NORTHWESTERN UNIVERSITY, Evanston, IL (US)

(72) Inventors: C. Shad Thaxton, Chicago, IL (US); Kaylin M. McMahon, Chicago, IL (US); Stephen E. Henrich, Chicago, IL (US)

(21) Appl. No.: 17/926,004

(22) PCT Filed: May 18, 2021
(86) PCT No.: PCT/US21/32930

§ 371 Date: Nov. 17, 2022

Related U.S. Application Data
(60) Provisional application No. 63/046,767, filed on Jul. 1, 2020.
(60) Provisional application No. 63/026,614, filed on May 18, 2020.

(51) Int. Cl. A61K 47/69; C12N 15/113; C07K 16/28; A61P 31/14

(52) U.S. Cl. CPC A61K 47/6917; A61K 47/6929; C12N 15/1138; C07K 16/28; A61P 31/14; C12N 2310/14

(57) ABSTRACT

Disclosed herein are agents that target cholesterol metabolism (e.g., synthetic nanostructures), pharmaceutical compositions, kits, or methods for treating and/or preventing viral infections. In some embodiments, the agents that target cholesterol metabolism and/or pharmaceutical compositions are delivered to the subject's respiratory system. In some embodiments, the viral infection is caused by a respiratory vims. In some embodiments, the virus is adenovirus (ADV); influenza virus, human bocavims (HBoV); human coronavirus (HCoV); human metapneumo vims (HMPV); human parainfluenza virus (HPIV); human respiratory syncytial vims (HRSV); human rhino vims (HRV); severe acute respiratory syndrome coronavirus (SARS-CoV); and Middle East Respiratory Syndrome coronavirus (MERS-CoV). In some embodiments, the virus is SARS-CoV-2.

### RELATED APPLICATIONS

This application claims the benefit under 35 U.S.C. § 119(e) of U.S. provisional application No. 63/026,614, filed May 18, 2020, and U.S. provisional application No. 63/046,767, filed on Jul. 1, 2020; the contents of each of which are incorporated by reference herein in its entirety.

### BACKGROUND

Nanoparticles are in the submicron size domain and possess unique size-dependent properties that make the materials superior compared to their bulk forms. The advanced chemical and physical properties associated with nanoparticles have led to their extensive use in the fields of biology and medicine. They have been shown to be useful for therapeutic, diagnostic, and research purposes.

Viral infections are a substantial cause of medical complications and can lead to a plethora of diseases. Of these viral respiratory infections are a leading cause of disease worldwide. Such viral respiratory infections can spread through a variety of means (e.g., contact, mucus, airborne droplets or particles) and affect individuals of all age groups and represent a serious threat to human health. The effects of infection on infected individuals can vary considerably and include completely asymptomatic manifestations, mild upper respiratory effects, and severe symptoms requiring hospitalization.¹ Understanding, preventing, and treating infections by these viruses is paramount in mitigating the effect of virally caused diseases. Accordingly, there is an ever-increasing need to find therapies to treat and prevent these disorders.

### SUMMARY

The present disclosure is based, at least in part, on compositions, kits, and methods for administering an agent that targets cholesterol metabolism such as a synthetic nanostructure (e.g., HDL-NP) that targets a cell surface receptor (e.g., CD36, SR-B1) in the respiratory system of a subject and are useful for treating a broad spectrum of virally associated diseases and bodily conditions (e.g., respiratory viruses).

In some aspects, the disclosure relates to a method for treating a viral infection in a subject, comprising administering to a subject having a viral infection, an agent that targets cholesterol metabolism (e.g., a synthetic HDL nanostructure) in an effective amount to inhibit viral entry into cells of the subject in order to treat the viral infection.

In some embodiments, the agent that targets cholesterol metabolism is delivered to the subject's respiratory system.

In some embodiments, the subject is identified as having a respiratory viral infection caused by a respiratory virus.

In some embodiments, the respiratory virus is selected from the group consisting of: adenovirus (ADV); influenza virus, human bocavirus (HBoV); human coronavirus (HCoV);

human metapneumovirus (HMPV); human parainfluenza virus (HPIV); human respiratory syncytial virus (HRSV); human rhinovirus (HRV); severe acute respiratory syndrome coronavirus (SARS-CoV); and Middle East Respiratory Syndrome coronavirus (MERS-CoV). In some embodiments, the respiratory virus is a coronavirus. In some embodiments, the coronavirus is a SARS-CoV or a MERS-CoV. In some embodiments, the respiratory virus is a respiratory syncytial virus.

In some embodiments, the subject is identified as having a viral infection with a virus that infects a scavenger receptor type B-1 (SR-B1), CD-36 receptor, low-density lipoprotein receptor (LDL-R), and/or Angiotensin-Converting Enzyme 2 (ACE2) positive cell.

In some aspects, the disclosure relates to a method for treating severe acute respiratory syndrome coronavirus 2 (SARS-CoV-2) infection in a subject, comprising administering to a subject infected with SARS-CoV-2 an agent that targets cholesterol metabolism in an effective amount to treat the SARS-CoV-2 infection in the subject.

In some embodiments, the agent that targets cholesterol metabolism is a synthetic HDL nanostructure, an inhibitory nucleic acid (e.g., antisense RNA, siRNA, microRNA, shRNA) that targets a cholesterol metabolism gene, or an antibody that inhibits the function of a protein encoded by a cholesterol metabolism gene.

In some embodiments, the cholesterol metabolism gene is scavenger receptor type B-1 (SR-B1), CD36, low-density lipoprotein receptor (LDL-R), or Angiotensin-Converting Enzyme 2 (ACE2). In some embodiments, the agent that targets cholesterol metabolism inhibits the function of a cell-surface receptor, optionally wherein the cell-surface receptor is scavenger receptor type B-1 (SR-B1), CD36, low-density lipoprotein receptor (LDL-R), or Angiotensin-Converting Enzyme 2 (ACE2).

In some embodiments, the synthetic HDL nanostructure of any of the methods herein, comprises a nanostructure core; an apolipoprotein; and a shell comprising a lipid surrounding and attached to the nanostructure core, wherein the shell comprises a phospholipid.

In some embodiments, the apolipoprotein of any of the synthetic HDL nano structures of any of the methods herein, is apolipoprotein A-I, apolipoprotein A-II, or apolipoprotein E.

In some embodiments, the nanostructure of any of the methods herein, further comprises a cholesterol.

In some embodiments, the shell of any of the synthetic HDL nanostructures of any of the methods herein, substantially surrounds the nanostructure core. In some embodiments, the shell comprises a lipid monolayer. In some embodiments, the shell comprises a lipid bilayer.

In some embodiments, at least a portion of the lipid bilayer of the shell of any of the synthetic HDL nanostructures of any of the methods herein, is covalently bound to the core.

In some embodiments, the core of any of the synthetic HDL nanostructures of any of the methods herein, has a largest cross-sectional dimension of less than or equal to about 5 nanometers (nm).

In some embodiments, the nanostructure core of any of the synthetic HDL nanostructures of any of the methods herein, is an inorganic nanostructure core. In some embodiments, the nanostructure core comprises gold. In some embodiments, the nanostructure core of any of the synthetic HDL nanostructures of any of the methods herein, is an organic nanostructure core.

In some embodiments, the synthetic HDL nanostructure of any of the methods herein, has a diameter of less than or equal to about 15 nanometers (nm).

In some embodiments, the inhibitory nucleic acid that targets a cholesterol metabolism gene is an siRNA (e.g., an siRNA that targets SR-B1).

In some embodiments, the antibody that inhibits the function of a protein encoded by a cholesterol metabolism gene is an anti-SR-B1 antibody (i.e., an antibody that specifically binds to SR-B1.

In some embodiments, in any of the methods disclosed herein, any of the agents that target cholesterol metabolism disclosed herein are administered to the subject once or twice a day. In some embodiments, in any of the methods disclosed herein, any of the agents that target cholesterol metabolism disclosed herein are administered to the subject once every other day.

In some embodiments, in any of the methods disclosed herein, any of the agents that target cholesterol metabolism disclosed herein are administered to the subject in combination with an anti-inflammatory agent.

In some embodiments, in any of the methods disclosed herein, any of the agents that target cholesterol metabolism disclosed herein are administered to the subject by intranasal administration.

In some embodiments, in any of the methods disclosed herein, any of the agents that target cholesterol metabolism disclosed herein are administered to the subject by oral administration with an oral dosage form that is a liquid, a spray or mist.

In some embodiments, in any of the methods disclosed herein, any of the agents that target cholesterol metabolism disclosed herein are administered to the subject by inhalation.

In some embodiments, in any of the methods disclosed herein, any of the agents that target cholesterol metabolism disclosed herein are administered to the subject systemically. In some embodiments, in any of the methods disclosed herein, any of the agents that target cholesterol metabolism disclosed herein are administered to the subject by intranasal administration.

In some embodiments, in any of the methods disclosed herein the subject is identified as having a comorbid disorder selected from the group consisting of hypertension, cardiovascular disease, obesity, and diabetes.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIG. 1 shows imaging using green fluorescent protein (GFP) of viral entry of VSV-G pseudotyped lentivirus into human hepatoma (HepG2) cells over a time period both in the absence and presence of HDL NPs. Top row of panels shows viral entry into the cells in the absence of HDL NPs over a period of 12 hours (h) (top left panel), 24 h (top middle panel), and 36 h (top right panel). Bottom row of panels shows viral entry into the cells in the presence of HDL NPs over a period of 12 hours (h) (bottom left panel), 24 h (bottom middle panel), and 36 h (bottom right panel). Viral load: 0.25 micrograms (μg) per milliliter (mL). HDL NP concentration: 50 nanomolar (nM).

FIGS. 2A-2D show graphs of the effect of HDL NPs on the number of human hepatoma (HepG2) cells infected by VSV-G pseudotyped lentivirus (expressing green fluorescence protein (GFP)) over time. FIG. 2A shows the number of virally infected cells over a period of time in three sample sets; Phosphate Buffered Saline (PBS); virus exposed without HDL NPs; and virus exposed with HDL NPs. Hours are shown on the x-axis; number of infected cells on the y-axis. FIG. 2B shows the number of infected cells of the exposed cells with HDL NPs sample as a percentage of number of infected cells of the exposed cells without HDL NPs sample after 48 h. Viral load: 0.25 μg/mL. HDL NP concentration: 50 nM. Time elapsed: 48 h. FIG. 2C shows the total integrated density of green fluorescence over time. FIG. 2D shows the total integrated density of green fluorescence after 48 h.

FIG. 3 shows imaging using green fluorescent protein (GFP) of viral entry of SARS-CoV-2-pseudotype lentivirus into human hepatoma (HepG2) cells over a time period both in the absence and presence of HDL NPs. Top row of panels shows viral entry into the cells in the absence of HDL NPs over a period of 12 hours (h) (top left panel), 24 h (top middle panel), and 36 h (top right panel). Bottom row of panels shows viral entry into the cells in the presence of HDL NPs over a period of 12 hours (h) (bottom left panel), 24 h (bottom middle panel), and 36 h (bottom right panel). Viral load: 0.5×10\hat{ }4 Foci Forming Units (FFU)/mL. HDL NP concentration: 50 nanomolar (nM).

FIGS. 4A-4D show graphs of the effect of HDL NPs on the number of human hepatoma (HepG2) cells infected by SARS-CoV-2-pseudotype lentivirus (expressing GFP) over time. FIG. 4A shows the number of virally infected cells over a period of time in four sample sets; Phosphate Buffered Saline (PBS); HDL NPs alone without virus; virus exposed without HDL NPs; and virus exposed with HDL NPs. Hours are shown on the x-axis; number of infected cells on the y-axis. FIG. 4B shows the number of infected cells after 48 h for the virus exposed group without HDL NP treatment compared to the group that received both virus exposure and HDL NP treatment. Viral load: 0.5×10\hat{ }4 Foci Forming Units (FFU)/mL. HDL NP concentration: 50 nM. Time elapsed: 48 h. FIG. 4C shows the total integrated density of green fluorescence over time. FIG. 4D shows the total integrated density of green fluorescence after 48 h.

FIG. 5 shows imaging using green fluorescent protein (GFP) of viral entry of SARS-CoV-2-pseudotype lentivirus into human embryonic kidney (HEK293) cells over a time period both in the absence and presence of HDL NPs. Top row of panels shows viral entry into the cells in the absence of HDL NPs over a period of 12 hours (h) (top left panel), 24 h (top middle panel), and 36 h (top right panel). Bottom row of panels shows viral entry into the cells in the presence of HDL NPs over a period of 12 hours (h) (bottom left panel), 24 h (bottom middle panel), and 36 h (bottom right panel). Viral load: 0.5×10\hat{ }4 Foci Forming Units (FFU)/mL. HDL NP concentration: 50 nanomolar (nM).

FIGS. 6A-6D show graphs of the effect of HDL NPs on the number of human embryonic kidney (HEK293) cells infected by SARS-CoV-2-pseudotype lentivirus (expressing GFP) over time. FIG. 6A shows the number of virally infected cells over a period of time in four sample sets; Phosphate Buffered Saline (PBS); HDL NPs alone without virus; virus exposed without HDL NPs; and virus exposed with HDL NPs. Hours are shown on the x-axis; number of infected cells on the y-axis. FIG. 6B shows the number of infected cells after 48 h for the virus exposed group without HDL NP treatment compared to the group that received both virus exposure and HDL NP treatment. Viral load: 0.5×10\hat{ }4 Foci Forming Units (FFU)/mL. HDL NP concentration: 50 nM. Time elapsed: 48 h. FIG. 6C shows the total integrated density of green fluorescence over time. FIG. 6D shows the total integrated density of green fluorescence after 48 h.

FIG. 7 shows western blot results for expression of scavenger receptor type B-1 (SR-B1). The left panel shows SR-B1 expression in HEK293 (ACE2 over-expressing) cells incubated with phosphate-buffered saline (PBS), scramble RNA control (siCntrl) or siRNA that targets SR-B1 (siSR-B1). The center panel shows SR-B1 expression in HepG2 cells incubated with PBS, scramble RNA control (siCntrl) or siRNA that targets SR-B1 (siSR-B1). The right panel shows SR-B1 expression in HEK293 and HepG2 cells under conditions where cells were treated with HDL NPs (50 nM) or PBS control. In all experiments, β-actin was used as a control for baseline expression.

FIG. 8 shows live cell imaging results of HEK293 (ACE2 over-expressing) cells infected with GFP-expressing SARS-CoV-2 pseudovirus. Cells were treated with (i) virus alone, (ii) virus+HDL NPs (50 nM), or (iii) virus, HDL NPs (50 nM) and SR-B1 siRNA (60 nM), for 48 h.

FIG. 9 shows quantification of live cell imaging results for experiments testing SR-B1 dependence. HEK293 cells were infected with GFP-expressing SARS-CoV-2 pseudovirus. Cells were treated with (i) virus+scramble RNA, (ii) virus+HDL NPs (50 nM)+scramble RNA, (iii) virus+SR-B1 siRNA, or (iv) virus+HDL NP+SR-B1 siRNA, for 48 h. GFP expression was quantified as total integrated density of green fluorescence per image. Left panel displays GFP expression over time. Right panel shows the GFP expression at the 48 h timepoint.

FIG. 10 shows live cell imaging results of HEK293 (ACE2 over-expressing) cells infected with GFP-expressing SARS-CoV-2 pseudovirus with (right panel) or without (left panel) co-treatment with an SR-B1 blocking antibody (20 μg/mL).

FIG. 11 shows quantification of live cell imaging results. HEK293 (ACE2 over-expressing) cells were infected with GFP-expressing SARS-CoV-2 pseudovirus with or without co-treatment with an SR-B1 blocking antibody (20 μg/mL). Data shown are at the 48 h time point. GFP expression was quantified using total integrated density of green fluorescence per image.

### DETAILED DESCRIPTION

The present invention relates to drugs (e.g., nanostructures, HDL-NPs) comprising agents that target (e.g., disrupt) cholesterol metabolism. In some embodiments, such agents are high density lipoproteins nanoparticles (HDL-NPs) that are useful for the treatment of viral infection in a subject. In other embodiments, such agents are inhibitory nucleic acids (e.g., antisense RNA, microRNA, siRNA) or inhibitory antibodies that target genes or proteins involved in cholesterol metabolism. The drugs of the present invention, when administered to the respiratory system (e.g., orally, intra-nasally, via aerosol or inhalant, as a liquid, as a spray, as a mist, topically to skin, eye, nose, airway, lung or iv) can drastically inhibit viral entry into cells, and proliferation and infection of the virus. These particles through their interaction with scavenger receptors in the respiratory system disrupt the viral attachment and/or entry mechanism and therefor can be used as a prophylactic or treatment for respiratory virus. These findings have tremendous implications for prevention of a plethora of diseases associated with respiratory infection which can lead to a number of negative health problems.

HDL target cells to modulate cell membrane and cellular cholesterol metabolism. The invention utilizes synthetic HDL nanostructures that mimic some features of native HDLs, but which have enhanced receptor binding properties and other desirable characteristics. Cholesterol homeostasis plays a critical role in a number of disease processes, including viral infection and inflammation. For instance, it has been demonstrated that cholesterol-rich and sphingolipid-rich raft microdomains in the plasma membrane are required for viral entry. Depletion of cell membrane cholesterol and modulation of lipid raft microdomains, in some embodiments, leads to drastic reduction in viral infectivity. Accordingly, targeting cholesterol metabolism in cells with agents as described herein are effective in reducing viral infections (e.g., SARs-CoV-2 infections).

Scavenger receptor type B-1 (SR-B1) is expressed by airway epithelial and immune cells. Reducing airway SR-B1 drastically attenuates the production of cytokines in the presence of innate immune cell stimulation. The agents of the invention (e.g., synthetic HDL NPs, inhibitory nucleic acids that target SR-B1, and inhibitory antibodies that specifically bind to SR-B1) are uniquely designed to target SR-B1, disrupt cholesterol-rich lipid rafts, and attenuate SR-B1 mediated cytokine production. Because the synthetic HDL NPs specifically target SR-B1 to modulate cell membrane and total cell cholesterol metabolism, they are useful for reducing the ability of a virus to enter cells. Data demonstrate that the agents of the invention (e.g. synthetic HDL NPs, inhibitory nucleic acids that target SR-B1, and inhibitory antibodies that specifically bind to SR-B1) inhibit viral entry into host cells.

The size and amphiphilic nature of the surface chemical composition of the synthetic HDL NP enables them to engage and tightly bind to SR-B1, expressed by inflammatory and epithelial cells lining the airway, to modulate cell membrane and cellular cholesterol, particularly in lipid raft cell membrane microdomains, to potently inhibit the entry of viruses. As such, this mechanism of action drastically reduce virus infectivity. Due to the surface composition of the synthetic HDL NPs, the surface bilayer may play a role in binding to the outer surface of the virus and prevent viral host cell interaction. Additionally, inhibition of viral entry by the synthetic HDL NPs, prevents a potent inflammatory host cell response that would be the result of viral entry.

The HDL-NPs and administration thereof, have a tremendous number of applications (e.g., preventing/treating diseases associated with respiratory viruses). Respiratory viruses that this drug may be useful for include, without limitation, adenovirus (ADV); influenza virus, human bocavirus (HBoV); human coronavirus (HCoV); human metapneumovirus (HMPV); human parainfluenza virus (HPIV); human respiratory syncytial virus (HRSV); human rhinovirus (HRV); severe acute respiratory syndrome coronavirus (SARS-CoV); and Middle East Respiratory Syndrome coronavirus (MERS-CoV), and the diseases associated therewith.

The present invention relates to a drug that can prevent and/or treat these conditions or diseases associated with respiratory viruses. Such a drug would meet tremendous needs across the spectrum of health care and would have a profound societal impact. To this end, the present disclosure provides methods for the administration of a synthetic nanostructure (e.g., HDL-NP) drug to treat or prevent any on the disorders disclosed herein. The HDL-NPs should bind to cell surface receptors (e.g., CD36, SR-B1) of cells of the respiratory system of a subject. The results herein suggest that, indeed, lipid nanoparticles (e.g., HDL-NPs) target cell receptors (e.g., CD36, SR-B1) of cells of the respiratory system and reveal these receptors as critical mediators of viral entry and infection of the cells. Interestingly, it was shown that viral entry and subsequent infection was reduced ˜68% in subjects exposed to respiratory virus in the presence of HDL NPs.

The compositions of the present disclosure allow for targeted delivery to the respiratory system when administered orally or intra-nasally. The compositions of the present disclosure are targeted at a cell surface receptor (e.g., CD36, SR-B1) expressed on cells of the respiratory system. In some embodiments, the compositions of the present disclosure comprise a synthetic nanostructure that is targeted (e.g., has the ability to bind) to SR-B1. In some embodiments, the compositions of the present disclosure comprise a synthetic nanostructure that is targeted (e.g., has the ability to bind) to CD36. In some embodiments, the compositions of the present disclosure comprise a synthetic nanostructure that is targeted (e.g., has the ability to bind) to low-density lipoprotein receptor (LDLR). In some embodiments, the synthetic nanostructure (e.g., HDL-NPs) is targeted to any one of the cell surface receptors in the respiratory system. In some embodiments, the synthetic nanostructure is targeted to SR-B1. In some embodiments, the synthetic nanostructure is targeted to CD36. The cores of the synthetic nanostructures (e.g., HDL-NPs) are preferably about 5 nanometers (nm) diameter nanostructures that are surface functionalized with phospholipids and apolipoprotein A-I.

Applications

As described herein, the methods and compositions of the present invention can be used to treat or prevent several diseases associated with viral infection (e.g., infection or disease resulting from viruses such as respiratory viruses, etc.). In some embodiments, the compositions of the present disclosure are used to treat or prevent a disease associated with, but not limited to, infection with any one or more of the following viruses: viral infectious diseases such as HIV, Cytomegalovirus, hepatitis A, B, C, D or E, herpes, herpes zoster (chicken pox), German measles (rubella virus), yellow fever, dengue (flavi viruses), influenza, Marburg or Ebola viruses, Japanese encephalitis virus, Western equine encephalitis virus, Haemophilus influenza type b (Hib), Meningitis, adenovirus infection, H5N1 influenza, severe acute respiratory syndrome (SARS), and H1N1 influenza. In some embodiments, the compositions of the present disclosure are used to treat or prevent a disease associated with, but not limited to, infection with any one or more of the following respiratory viruses: adenovirus (ADV); influenza virus, human bocavirus (HBoV); human coronavirus (HCoV); human metapneumovirus (HMPV); human parainfluenza virus (HPIV); human respiratory syncytial virus (HRSV); human rhinovirus (HRV); severe acute respiratory syndrome coronavirus (SARS-CoV); and Middle East Respiratory Syndrome coronavirus (MERS-CoV). In some embodiments, the respiratory virus is a SARS-CoV or MERS-CoV virus. In some embodiments, the virus is a coronavirus. In some embodiments, the infection or disease is associated with SARS-CoV-2.

In some embodiments, a subject is identified as having a respiratory viral disorder. The presence of a respiratory viral disorder may be assessed using any routine screening tests known in the art including tests for presence of viral particles as well as identification of symptoms such as lung inflammation.

In some embodiments the subject is further identified as having a comorbid disorder. In some embodiments the subject is further identified as having a comorbid disorder selected from the group consisting of hypertension, cardiovascular disease, obesity, and diabetes. In other embodiments the subject is further identified as not having a comorbid disorder. In other embodiments the subject is further identified as not having a comorbid disorder selected from the group consisting of hypertension, cardiovascular disease, obesity, and diabetes.

Synthetic Nanostructures

In some embodiments of the present disclosure, a synthetic HDL nanostructure is administered to the respiratory system for the treatment of the conditions disclosed herein. The synthetic nanostructure may be any synthetic HDL nanostructure having the property of being able to be bind to a cell surface receptor in the respiratory system (e.g., CD36, SR-B1). The synthetic HDL nanostructure may comprise a nanostructure core, a shell, the shell comprising a lipid layer surrounding and attached to the nanostructure core, and a protein associate with the shell. Examples of synthetic nanostructures useful for the present purposes are described below. In preferred embodiments, the synthetic HDL nanostructure may be a synthetic cholesterol binding nanostructure, i.e., a biomimic of mature, spherical HDL, e.g., in terms of the size, shape, surface chemistry and/or function of the structures. Control of such features may be accomplished at least in part by using a synthetic template for the formation of the nanostructures. For example, high-density lipoprotein synthetic nanoparticles (HDL-NP) may be formed by using a solid core NP such as a gold nanoparticle (Au-NP) (or other suitable entity or material) as a synthetic template to which other components (e.g., lipids, proteins, etc.) can be added.

Examples of synthetic nanostructures that can be used in the methods are described herein. The structure (e.g., HDL-NP) has a core and a shell surrounding the core. In embodiments in which the core is a nanostructure, the core includes a surface to which one or more components can be optionally attached. For instance, in some cases, core is a nanostructure surrounded by shell, which includes an inner surface and an outer surface. The shell may be formed, at least in part, of one or more components, such as a plurality of lipids, which may optionally associate with one another and/or with surface of the core. For example, components may be associated with the core by being covalently attached to the core, physisorbed, chemisorbed, or attached to the core through ionic interactions, hydrophobic and/or hydrophilic interactions, electrostatic interactions, van der Waals interactions, or combinations thereof. In one particular embodiment, the core includes a gold nanostructure and the shell is attached to the core through a gold-thiol bond.

Optionally, components can be crosslinked to one another. Crosslinking of components of a shell can, for example, allow the control of transport of species into the shell, or between an area exterior to the shell and an area interior of the shell. For example, relatively high amounts of crosslinking may allow certain small, but not large, molecules to pass into or through the shell, whereas relatively low or no crosslinking can allow larger molecules to pass into or through the shell. Additionally, the components forming the shell may be in the form of a monolayer or a multilayer, which can also facilitate or impede the transport or sequestering of molecules. In one exemplary embodiment, shell includes a lipid bilayer that is arranged to sequester cholesterol and/or control cholesterol efflux out of cells, as described herein.

It should be understood that a shell that surrounds a core need not completely surround the core, although such embodiments may be possible. For example, the shell may surround at least 50% (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more) of the surface area of a core. In some cases, the shell substantially surrounds a core. In other cases, the shell completely surrounds a core. The components of the shell may be distributed evenly across a surface of the core in some cases, and unevenly in other cases. For example, the shell may include portions (e.g., holes) that do not include any material in some cases. If desired, the shell may be designed to allow penetration and/or transport of certain molecules and components into or out of the shell, but may prevent penetration and/or transport of other molecules and components into or out of the shell. The ability of certain molecules to penetrate and/or be transported into and/or across a shell may depend on, for example, the packing density of the components forming the shell and the chemical and physical properties of the components forming the shell. As described herein, the shell may include one layer of material, or multilayers of materials in some embodiments.

In certain embodiments that synthetic nanostructure may further include one or more agents, such as a therapeutic or diagnostic agent. The agent may be a diagnostic agent (which may also be known as an imaging agent), a therapeutic agent, or both a diagnostic agent and a therapeutic agent. In certain embodiments the diagnostic agent is a tracer lipid. Tracer lipids may comprise a chromophore, a biotin subunit, or both a chromophore and a biotin subunit. The synthetic nanostructures (e.g., HDL NPs) can also be functionalized with other types of cargo such as nucleic acids. In certain embodiments the therapeutic agent may be a nucleic acid, antiviral agent or anti-inflammatory agent.

The one or more agents may be associated with the core, the shell, or both; e.g., they may be associated with surface of the core, inner surface of the shell, outer surface of the shell, and/or embedded in the shell. For example, one or more agents may be associated with the core, the shell, or both through covalent bonds, physisorption, chemisorption, or attached through ionic interactions, hydrophobic and/or hydrophilic interactions, electrostatic interactions, van der Waals interactions, or combinations thereof.

In some cases, the synthetic nanostructure is a synthetic cholesterol binding nanostructure having a binding constant (Kd) for cholesterol. In some embodiments, Kd is less than or equal to about 100 μM, less than or equal to about 10 μM, less than or equal to about 1 μM, less than or equal to about 0.1 μM, less than or equal to about 10 nM, less than or equal to about 7 nM, less than or equal to about 5 nM, less than or equal to about 2 nM, less than or equal to about 1 nM, less than or equal to about 0.1 nM, less than or equal to about 10 pM, less than or equal to about 1 pM, less than or equal to about 0.1 pM, less than or equal to about 10 fM, or less than or equal to about 1 fM. Methods for determining the amount of cholesterol sequestered and binding constants are known in the art.

The core of the nanostructure whether being a nanostructure core or a hollow core, may have any suitable shape and/or size. For instance, the core may be substantially spherical, non-spherical, oval, rod-shaped, pyramidal, cube-like, disk-shaped, wire-like, or irregularly shaped. In some embodiments, the core comprises a substantially spherical shape. In some embodiments, the core comprises a substantially non-spherical shape. In some embodiments, the core comprises a substantially oval shape. In some embodiments, the core comprises a substantially rod-like shape. In some embodiments, the core comprises a substantially pyramidal shape. In some embodiments, the core comprises a substantially cube-like shape. In some embodiments, the core comprises a substantially disk-like shape. In some embodiments, the core comprises a substantially wire-like shape. In some embodiments, the core comprises a substantially irregular shape. In preferred embodiments of the present invention, the core is less than or equal to about 5 nm in diameter. The core (e.g., a nanostructure core or a hollow core) may have a largest cross-sectional dimension (or, sometimes, a smallest cross-section dimension, or diameter) of, for example, less than or equal to about 500 nm, less than or equal to about 250 nm, less than or equal to about 100 nm, less than or equal to about 75 nm, less than or equal to about 50 nm, less than or equal to about 40 nm, less than or equal to about 35 nm, less than or equal to about 30 nm, less than or equal to about 25 nm, less than or equal to about 20 nm, less than or equal to about 15 nm, less than or equal to about 10 nm, less than or equal to about 5 nm, less than or equal to about 4 nm, less than or equal to about 3 nm, less than or equal to about 2 nm or less than or equal to about 1 nm. In some cases, the core has an aspect ratio of greater than about 1:1, greater than 3:1, or greater than 5:1. As used herein, “aspect ratio” refers to the ratio of a length to a width, where length and width measured perpendicular to one another, and the length refers to the longest linearly measured dimension.

In embodiments in which core includes a nanostructure core, the nanostructure core may be formed from any suitable material. In preferred embodiments, the core is formed from gold (e.g., made of gold (Au)). In some embodiments, the core is formed of a synthetic material (e.g., a material that is not naturally occurring, or naturally present in the body). In one embodiment, a nanostructure core comprises or is formed of an inorganic material. In some embodiments, a nanostructure core comprises or is formed of an organic material. The inorganic material may include, for example, a metal (e.g., Ag, Au, Pt, Fe, Cr, Co, Ni, Cu, Zn, and other transition metals), a semiconductor (e.g., silicon, silicon compounds and alloys, cadmium selenide, cadmium sulfide, indium arsenide, and indium phosphide), or an insulator (e.g., ceramics such as silicon oxide). The inorganic material may be present in the core in any suitable amount, e.g., at least 1 percent by weight (i.e., 1 wt %), 5 wt %, 10 wt %, 25 wt %, 50 wt %, 75 wt %, 90 wt %, or 99 wt %. In one embodiment, the core is formed of 100 wt % inorganic material. The nanostructure core may, in some cases, be in the form of a quantum dot, a carbon nanotube, a carbon nanowire, or a carbon nanorod. In some cases, the nanostructure core comprises, or is formed of, a material that is not of biological origin. In some embodiments, a nanostructure includes or may be formed of one or more organic materials such as a synthetic polymer and/or a natural polymer. Examples of synthetic polymers include non-degradable polymers such as polymethacrylate and degradable polymers such as polylactic acid, polyglycolic acid and copolymers thereof. Examples of natural polymers include hyaluronic acid, chitosan, and collagen.

Furthermore, a shell of a structure can have any suitable thickness. For example, the thickness of a shell may be at least 10 Angstroms, at least 0.1 nm, at least 1 nm, at least 2 nm, at least 5 nm, at least 7 nm, at least 10 nm, at least 15 nm, at least 20 nm, at least 30 nm, at least 50 nm, at least 100 nm, or at least 200 nm (e.g., from the inner surface to the outer surface of the shell). In some cases, the thickness of a shell is less than 200 nm, less than 100 nm, less than 50 nm, less than 30 nm, less than 20 nm, less than 15 nm, less than 10 nm, less than 7 nm, less than 5 nm, less than 3 nm, less than 2 nm, or less than 1 nm (e.g., from the inner surface to the outer surface of the shell). Such thicknesses may be determined prior to or after sequestration of molecules as described herein.

Those of ordinary skill in the art are familiar with techniques to determine sizes of structures and particles. Examples of suitable techniques include dynamic light scattering (DLS) (e.g., using a Malvern Zetasizer instrument), transmission electron microscopy, scanning electron microscopy, electroresistance counting and laser diffraction. Other suitable techniques are known to those or ordinary skill in the art. Although many methods for determining sizes of nanostructures are known, the sizes described herein (e.g., largest or smallest cross-sectional dimensions, thicknesses) refer to ones measured by dynamic light scattering.

The shell of a structure described herein may comprise any suitable material, such as a hydrophobic material, a hydrophilic material, and/or an amphiphilic material. Although the shell may include one or more inorganic materials such as those listed above for the nanostructure core, in many embodiments the shell includes an organic material such as a lipid or certain polymers. The components of the shell may be chosen, in some embodiments, to facilitate the sequestering of cholesterol or other molecules. For instance, cholesterol (or other sequestered molecules) may bind or otherwise associate with a surface of the shell, or the shell may include components that allow the cholesterol to be internalized by the structure. Cholesterol (or other sequestered molecules) may also be embedded in a shell, within a layer or between two layers forming the shell.

The components of a shell may be charged, e.g., to impart a charge on the surface of the structure, or uncharged. In some embodiments, the surface of the shell may have a zeta potential of greater than or equal to about −75 mV, greater than or equal to about −60 mV, greater than or equal to about −50 mV, greater than or equal to about −40 mV, greater than or equal to about −30 mV, greater than or equal to about −20 mV, greater than or equal to about −10 mV, greater than or equal to about 0 mV, greater than or equal to about 10 mV, greater than or equal to about 20 mV, greater than or equal to about 30 mV, greater than or equal to about 40 mV, greater than or equal to about 50 mV, greater than or equal to about 60 mV, or greater than or equal to about 75 mV. The surface of the shell may have a zeta potential of less than or equal to about 75 mV, less than or equal to about 60 mV, less than or equal to about 50 mV, less than or equal to about 40 mV, less than or equal to about 30 mV, less than or equal to about 20 mV, less than or equal to about 10 mV, less than or equal to about 0 mV, less than or equal to about −10 mV, less than or equal to about −20 mV, less than or equal to about −30 mV, less than or equal to about −40 mV, less than or equal to about −50 mV, less than or equal to about −60 mV, or less than or equal to about −75 mV. Other ranges are also possible. Combinations of the above-referenced ranges are also possible (e.g., greater than or equal to about −60 mV and less than or equal to about −20 mV). As described herein, the surface charge of the shell may be tailored by varying the surface chemistry and components of the shell.

In one set of embodiments, a structure described herein or a portion thereof, such as a shell of a structure, includes one or more natural or synthetic lipids or lipid analogs (i.e., lipophilic molecules). One or more lipids and/or lipid analogues may form a single layer or a multi-layer (e.g., a bilayer) of a structure. In some instances where multi-layers are formed, the natural or synthetic lipids or lipid analogs interdigitate (e.g., between different layers). Non-limiting examples of natural or synthetic lipids or lipid analogs include fatty acyls, glycerolipids, glycerophospholipids, sphingolipids, saccharolipids and polyketides (derived from condensation of ketoacyl subunits), and sterol lipids and prenol lipids (derived from condensation of isoprene subunits).

In one particular set of embodiments, a structure described herein includes one or more phospholipids. The one or more phospholipids may include, for example, phosphatidylcholine, phosphatidylglycerol, lecithin, β,γ-dipalmitoyl-α-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, 1,2-dipalmitoyl-sn-glycero-3-phosphothioethanol, and combinations thereof. In some cases, a shell (e.g., a bilayer) of a structure includes 50-200 natural or synthetic lipids or lipid analogs (e.g., phospholipids). For example, the shell may include less than about 500, less than about 400, less than about 300, less than about 200, or less than about 100 natural or synthetic lipids or lipid analogs (e.g., phospholipids), e.g., depending on the size of the structure.

Non-phosphorus containing lipids may also be used such as stearylamine, docecylamine, acetyl palmitate, and fatty acid amides. In other embodiments, other lipids such as fats, oils, waxes, cholesterol, sterols, fat-soluble vitamins (e.g., vitamins A, D, E and K), glycerides (e.g., monoglycerides, diglycerides, triglycerides) can be used to form portions of a structure described herein.

A portion of a structure described herein such as a shell or a surface of a nanostructure may optionally include one or more alkyl groups, e.g., an alkane-, alkene-, or alkyne-containing species that optionally imparts hydrophobicity to the structure. An “alkyl” group refers to a saturated aliphatic group, including a straight-chain alkyl group, branched-chain alkyl group, cycloalkyl (alicyclic) group, alkyl substituted cycloalkyl group, and cycloalkyl substituted alkyl group. The alkyl group may have various carbon numbers, e.g., between C2 and C40, and in some embodiments may be greater than C5, C10, C15, C20, C25, C30, or C35. In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C1-C12 for straight chain, C3-C12 for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The alkyl group may include any suitable end group, e.g., a thiol group, an amino group (e.g., an unsubstituted or substituted amine), an amide group, an imine group, a carboxyl group, or a sulfate group, which may, for example, allow attachment of a ligand to a nanostructure core directly or via a linker. For example, where inert metals are used to form a nanostructure core, the alkyl species may include a thiol group to form a metal-thiol bond. In some instances, the alkyl species includes at least a second end group. For example, the species may be bound to a hydrophilic moiety such as polyethylene glycol. In other embodiments, the second end group may be a reactive group that can covalently attach to another functional group. In some instances, the second end group can participate in a ligand/receptor interaction (e.g., biotin/streptavidin).

In some embodiments, the shell includes a polymer. For example, an amphiphilic polymer may be used. The polymer may be a diblock copolymer, a triblock copolymer, etc., e.g., where one block is a hydrophobic polymer and another block is a hydrophilic polymer. For example, the polymer may be a copolymer of an α-hydroxy acid (e.g., lactic acid) and polyethylene glycol. In some cases, a shell includes a hydrophobic polymer, such as polymers that may include certain acrylics, amides and imides, carbonates, dienes, esters, ethers, fluorocarbons, olefins, styrenes, vinyl acetals, vinyl and vinylidene chlorides, vinyl esters, vinyl ethers and ketones, and vinylpyridine and vinylpyrrolidones polymers. In other cases, a shell includes a hydrophilic polymer, such as polymers including certain acrylics, amines, ethers, styrenes, vinyl acids, and vinyl alcohols. The polymer may be charged or uncharged. As noted herein, the particular components of the shell can be chosen so as to impart certain functionality to the structures.

Where a shell includes an amphiphilic material, the material can be arranged in any suitable manner with respect to the nanostructure core and/or with each other. For instance, the amphiphilic material may include a hydrophilic group that points towards the core and a hydrophobic group that extends away from the core, or the amphiphilic material may include a hydrophobic group that points towards the core and a hydrophilic group that extends away from the core. Bilayers of each configuration can also be formed.

The structures described herein may also include one or more proteins, polypeptides and/or peptides (e.g., synthetic peptides, amphiphilic peptides). In one set of embodiments, the structures include proteins, polypeptides and/or peptides that can increase the rate of cholesterol transfer or the cholesterol-carrying capacity of the structures. The one or more proteins or peptides may be associated with the core (e.g., a surface of the core or embedded in the core), the shell (e.g., an inner and/or outer surface of the shell, and/or embedded in the shell), or both. Associations may include covalent or non-covalent interactions (e.g., hydrophobic and/or hydrophilic interactions, electrostatic interactions, van der Waals interactions).

An example of a suitable protein that may associate with a structure described herein is an apolipoprotein, such as apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Specifically, apo A1, apo A2, and apo E promote transfer of cholesterol and cholesteryl esters to the liver for metabolism and may be useful to include in structures described herein. Additionally or alternatively, a structure described herein may include one or more peptide analogues of an apolipoprotein, such as one described above. A structure may include any suitable number of, e.g., at least 1, 2, 3, 4, 5, 6, or 10, apolipoproteins or analogues thereof. In certain embodiments, a structure includes 1-6 apolipoproteins, similar to a naturally occurring HDL particle. Of course, other proteins (e.g., non-apolipoproteins) can also be included in structures described herein.

It should be understood that the components described herein, such as the lipids, phospholipids, alkyl groups, polymers, proteins, polypeptides, peptides, enzymes, bioactive agents, nucleic acids, and species for targeting described above (which may be optional), may be associated with a structure in any suitable manner and with any suitable portion of the structure, e.g., the core, the shell, or both. For example, one or more such components may be associated with a surface of a core, an interior of a core, an inner surface of a shell, an outer surface of a shell, and/or embedded in a shell. Furthermore, such components can be used, in some embodiments, to facilitate the sequestration, exchange and/or transport of materials (e.g., proteins, peptides, polypeptides, nucleic acids, nutrients) from one or more components of a subject (e.g., cells, tissues, organs, particles, fluids (e.g., blood), and portions thereof) to a structure described herein, and/or from the structure to the one or more components of the subject. In some cases, the components have chemical and/or physical properties that allow favorable interaction (e.g., binding, adsorption, transport) with the one or more materials from the subject.

In some aspects, the synthetic HDL-NP is in the form of an anionic nanostructure, comprising an HDL-NP (inert core, a lipid shell surrounding the inert core, and an apolipoprotein functionalized to the inert core) complexed with a cationic lipid-nucleic acid complex comprised of a nucleic acid sequence. The cationic lipid-nucleic acid complex and anionic nanostructure has a negative ζ-potential. In such a configuration, the HDL-NP may be referred to herein as a templated lipoprotein particle (TLP). A TLP, in some embodiments forms an anionic nanostructure aggregate with RNA or DNA in single or double stranded form. Thus, in some embodiments the TLP is comprised of single stranded or double stranded RNA complexed with a cationic lipid. In some embodiments each strand of a duplex RNA or DNA is conjugated separately to a cationic lipid. In some embodiments the RNA is not chemically modified. In other embodiments it is chemically modified. In some embodiments the inert core is a metal such as gold. In some embodiments the phospholipids are 1,2-dioleoyl-sn-glycero-3-phophocholine (DOPC) and 1,2-dipalmitoyl-sn-glycero-3-phosphoethanolamine-N-[3-(2-pyridyldithio)propionate] (PDP-PE). In some embodiments the nanostructure comprises alternating layers of 1,2-dioleoyl-3-trimethylammonium-propane (DOTAP) and RNA.

In some embodiments, the nanostructure includes a cationic lipid. The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro- -3 aH-cyclopenta[d][1,3]dioxol-5-amine, (6Z,9Z,28Z,31Z)-heptatriaconta-6,9,28,31-tetraen-19-yl-4-(dimethylamino)butanoate, or a mixture thereof.

Other cationic lipids, which carry a net positive charge at about physiological pH, in addition to those specifically described above, may also be included in the lipid nanoparticle. Such cationic lipids include, but are not limited to, N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy trimethylaminopropane chloride salt (“DOTAP.Cl”); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), and 1,2-dioleoyl-sn-glycero-3-phosphocholine (“DOPC”).

In some aspects of the disclosure, the nanostructure comprises a cationic lipid (e.g., DOTAP) is mixed with a nucleic acid (e.g., RNA) in a molar ratio of about 1:1, of about 2:1, of about 3:1, of about 4:1, of about 5:1, of about 6:1, of about 7:1, of about 8:1, of about 9:1, of about 10:1, of about 11:1, of about 12:1, of about 13:1, of about 14:1, of about 15:1, of about 16:1, of about 17:1, of about 18:1, of about 19:1, of about 20:1, of about 21:1, of about 22:1, of about 23:1, of about 24:1, of about 25:1, of about 26:1, of about 27:1, of about 28:1, of about 29:1, of about 30:1, of about 31:1, of about 32:1, of about 33:1, of about 34:1, of about 35:1, of about 36:1, of about 37:1, of about 38:1, of about 39:1, of about 40:1, of about 41:1, of about 42:1, of about 43:1, of about 44:1, of about 45:1, of about 46:1, of about 47:1, of about 48:1, of about 49:1, of about 50:1, of about 60:1, of about 70:1, of about 80:1, of about 90:1, or of about 100:1. In some embodiments, the cationic lipid (e.g. DOTAP) is mixed with the nucleic acid (e.g., RNA) in a molar ratio of 10:1, 20:1, 30:1 or 40:1.

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine, monophosphoryl lipid A (MPLA), or glycopyranoside lipid A (GLA). In some embodiments, the nanostructures (e.g., HDL NP, TLP) of the disclosure comprise apolipoprotein. The apolipoprotein can be apolipoprotein A (e.g., apo A-I, apo A-II, apo A-IV, and apo A-V), apolipoprotein B (e.g., apo B48 and apo B100), apolipoprotein C (e.g., apo C-I, apo C-II, apo C-III, and apo C-IV), and apolipoproteins D, E, and H. Additionally, a structure described herein may include one or more peptide analogues of an apolipoprotein, such as one described above. Of course, other proteins (e.g., non-apolipoproteins) can also be included in the nanostructures described herein. In some embodiments, the nanostructure of the present disclosure contain apolipoprotein A-I (apoA-I), which is the main protein constituent of HDLs. The nanostructures of the present disclosure are able to bind with high affinity to SCARB1. The nanostructures of the present disclosure have reduced toxicity. In some embodiments, the apolipoprotein is apolipoprotein A-I.

The surface density of bound oligonucleotides to the structures may also be controlled. Oligonucleotides such as DNA, RNA, or siRNA may be attached to a nanostructure core using techniques such as electrostatic adsorption or chemisorption techniques, for example, Au—SH conjugation chemistry.

Inhibitory Nucleic Acids

An agent that targets (e.g., disrupts) cholesterol metabolism may be an inhibitory nucleic acid that causes specific gene knockdown of a gene involved in cholesterol metabolism (a “cholesterol metabolism gene”). In some embodiments, a cholesterol metabolism gene encodes scavenger receptor type B-1 (SR-B1), CD36, low-density lipoprotein receptor (LDL-R), or Angiotensin-Converting Enzyme 2 (ACE2). An inhibitory nucleic acid may specifically inhibit the expression and/or function of scavenger receptor type B-1 (SR-B1), CD36, low-density lipoprotein receptor (LDL-R), or Angiotensin-Converting Enzyme 2 (ACE2).

Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA expression constructs) are used to reduce expression of a gene in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a specific promoter operably linked to a gene encoding the small interfering nucleic acid.

In some aspects, the nucleic acid or oligonucleotide regulate the expression of the cholesterol metabolism gene. As used herein, “regulating gene expression” or “gene regulation” are used interchangeably and includes a wide range of mechanisms that are used by cells to increase or decrease the production of specific gene products (e.g., protein, RNA, etc.). In some embodiments the nucleic acid or oligonucleotide is an inhibitory nucleic acid. The inhibitory nucleic acid may be, for instance, an siRNA or an antisense molecule that inhibits expression of a protein that will have a therapeutic effect. The inhibitory nucleic acids may be designed using routine methods in the art.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to nucleic acids or oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006)). siRNAs with amide-linked oligoribonucleosides have been generated that are more resistant to S1 nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006).

Other molecules that can be used to inhibit expression of a gene include antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996).

Other inhibitor molecules that can be used include antisense nucleic acids (single or double stranded). Antisense nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

An inhibitory nucleic acid useful in the invention will generally be designed to have partial or complete complementarity with one or more target genes (e.g., cholesterol metabolism gene). The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

An inhibitory nucleic acid can be single stranded or double stranded. A double stranded oligonucleotide is also referred to herein as a duplex. Double-stranded oligonucleotides of the invention can comprise two separate complementary nucleic acid strands. The nucleic acids of the invention are synthetic or isolated nucleic acids. As used herein, “duplex” includes a double-stranded nucleic acid molecule(s) in which complementary sequences are hydrogen bonded to each other. The complementary sequences can include a sense strand and an antisense strand. The antisense nucleotide sequence can be identical or sufficiently identical to the target gene to mediate effective target gene inhibition (e.g., at least about 98% identical, 96% identical, 94%, 90% identical, 85% identical, or 80% identical) to the target gene sequence.

A double-stranded nucleic acid or oligonucleotide can be double-stranded over its entire length, meaning it has no overhanging single-stranded sequences and is thus blunt-ended. In other embodiments, the two strands of the double-stranded polynucleotide can have different lengths producing one or more single-stranded overhangs. A double-stranded polynucleotide of the invention can contain mismatches and/or loops or bulges. In some embodiments, it is double-stranded over at least about 70%, 80%, 90%, 95%, 96%, 97%, 98% or 99% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

Nucleic acids or oligonucleotides associated with the invention can be modified such as at the sugar moiety, the phosphodiester linkage, and/or the base. As used herein, “sugar moieties” includes natural, unmodified sugars, including pentose, ribose and deoxyribose, modified sugars and sugar analogs. Modifications of sugar moieties can include replacement of a hydroxyl group with a halogen, a heteroatom, or an aliphatic group, and can include functionalization of the hydroxyl group as, for example, an ether, amine or thiol.

Modification of sugar moieties can include 2′-O-methyl nucleotides, which are referred to as “methylated.” In some instances, polynucleotides associated with the invention may only contain modified or unmodified sugar moieties, while in other instances, polynucleotides contain some sugar moieties that are modified and some that are not. In some instances, modified nucleomonomers include sugar- or backbone-modified ribonucleotides. Modified ribonucleotides can contain a non-naturally occurring base such as uridines or cytidines modified at the 5′-position, e.g., 5′-(2-amino)propyl uridine and 5′-bromo uridine; adeno sines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides can have the 2′-OH group replaced by an H, alkoxy (or OR), R or alkyl, halogen, SH, SR, amino (such as NH2, NHR, NR2), or CN group, wherein R is lower alkyl, alkenyl, or alkynyl. In some embodiments, modified ribonucleotides can have the phosphodiester group connecting to adjacent ribonucleotides replaced by a modified group, such as a phosphorothioate group.

In some aspects, 2′-O-methyl modifications can be beneficial for reducing undesirable cellular stress responses, such as the interferon response to double-stranded nucleic acids. Modified sugars can include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH2CH═CH2), 2′-propargyl, 2′-propyl, ethynyl, ethenyl, propenyl, and cyano and the like. The sugar moiety can also be a hexose.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N6-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

Antibodies

An agent that targets (e.g., disrupts) cholesterol metabolism may be an antibody that binds (e.g., specifically binds) to a protein encoded by a cholesterol metabolism gene. In some embodiments, a cholesterol metabolism gene encodes scavenger receptor type B-1 (SR-B1), CD36, low-density lipoprotein receptor (LDL-R), or Angiotensin-Converting Enzyme 2 (ACE2). An antibody may bind (e.g., specifically bind such that it inhibits protein function) to scavenger receptor type B-1 (SR-B1), CD36, low-density lipoprotein receptor (LDL-R), or Angiotensin-Converting Enzyme 2 (ACE2).

In some embodiments, the antibody specifically binds the protein encoded by a cholesterol metabolism gene (e.g., SR-B1). As used herein, “specifically binds,” refers to an antibody which binds to the protein encoded by a cholesterol metabolism gene (e.g., SR-B1) with greater affinity, avidity, more readily, and/or with greater duration than it binds to another molecule (e.g., an off-target molecule). In some embodiments, the antibody binds the protein encoded by a cholesterol metabolism gene (e.g., SR-B1) covalently. In some embodiments, the antibody binds the protein encoded by a cholesterol metabolism gene (e.g., an antigen of SR-B1) non-covalently.

In some embodiments, the antibody described herein is a monoclonal antibody, a chimeric antibody, a humanized antibody, a human engineered antibody, a human antibody, a single chain antibody (scFv), or an antibody fragment.

As used herein, “antibody” refers to a polypeptide of the immunoglobulin family that is capable of binding to a corresponding antigen on a protein encoded by a cholesterol metabolism gene. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies include, but are not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure). The antibodies can be of any isotype/class (e.g., IgG, IgE, IgM, IgD, IgA and IgY), or subclass (e.g., IgG1, IgG2, IgG3, IgG4, IgA1 and IgA2).

As used herein, “complementarity-determining domains” or “complementary-determining regions” (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains.

CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W.H. Freeman & Co., New York, 2000).

The positions of the CDRs and framework regions can be determined using various well known definitions in the art, e.g., Kabat, Chothia, and AbM (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HC CDR1), 50-65 (HC CDR2), and 95-102 (HC CDR3) in a VH, e.g., a mammalian VH, e.g., a human VH; and amino acid residues 24-34 (LC CDR1), 50-56 (LC CDR2), and 89-97 (LC CDR3) in a VL, e.g., a mammalian VL, e.g., a human VL.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

In some embodiments, the antibody is an antibody fragment or antigen-binding fragment. An antibody fragment is protein or polypeptide derived from an antibody. An antigen-binding fragment is a protein or polypeptide derived from an antibody that is capable of binding to the same epitope or antigen as the antibody from which it was derived.

Pharmaceutical Compositions

As described herein, the synthetic nanostructures (e.g., HDL NP, TLP) may be used in “pharmaceutical compositions” or “pharmaceutically acceptable” compositions (also referred to as drugs), which comprise a therapeutically effective amount of one or more of the structures described herein, formulated together with one or more pharmaceutically acceptable carriers, additives, and/or diluents. The pharmaceutical compositions described herein may be useful for treating viruses, respiratory viruses, diseases associated therewith, or other conditions. It should be understood that any suitable structures described herein can be used in such pharmaceutical compositions, including those described in connection with the figures. In some cases, the structures in a pharmaceutical composition have a nanostructure core comprising an inorganic material and a shell substantially surrounding and attached to the nanostructure core. In some embodiments, the structures in a pharmaceutical composition have a nanostructure core comprising an organic material and a shell substantially surrounding and attached to the nanostructure core.

The pharmaceutical compositions may be specially formulated for administration in solid or liquid form, including those adapted for the following: administration to the respiratory system, including, intra-nasal administration, for example, liquids, sprays, mists, aerosols, or inhalants powders, oral administration, for example, liquids, sprays, mists, aerosols, or inhalants, drenches (aqueous or non-aqueous solutions or suspensions), tablets, e.g., those targeted for buccal, and sublingual, boluses, powders, granules, pastes for application to the tongue; as a sterile solution or suspension, or sustained-release formulation; spray applied to the oral cavity; for example, as cream or foam. In some embodiments, the liquid or solid may be a composition or formulation for use in a nebulizer or other device which transforms the composition or formulation into a form for administration to the respiratory system. In some instances, the composition may be in the form of a solid, which is released in the oral or nasal cavity for release into the respiratory system. In some embodiment, the release may be triggered by contact with the saliva of the cavity, in some embodiments, the release may be triggered by pressure (e.g., applied force by fingers, tissues, teeth, tongue, lips, etc.).

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Pharmaceutical compositions described herein include those suitable for administration to the respiratory system. The formulations may conveniently be presented in unit dosage form and may be prepared by any methods well known in the art of pharmacy. The amount of active ingredient that can be combined with a carrier material to produce a single dosage form will vary depending upon the host being treated, and the particular mode of administration. The amount of active ingredient that can be combined with a carrier material to produce a single dosage form will generally be that amount of the compound that produces a therapeutic effect. Generally, this amount will range from about 1% to about 99% of active ingredient, from about 5% to about 70%, or from about 10% to about 30%.

The compositions of the present disclosure (e.g., HDL-NPs, synthetic nanostructures) suitable for intra-nasal administration may be in the form of liquid, sprays, mists, powders, inhalants, aerosols, granules, or other formulations which facilitate administration to the respiratory system via nasal administration. The compositions of the present disclosure (e.g., HDL-NPs, synthetic nanostructures) suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a structure described herein as an active ingredient.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered structure is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or in a certain portion of the respiratory system, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the structures described herein include pharmaceutically acceptable emulsions, microemulsions, solutions, dispersions, suspensions, syrups and elixirs. In addition to the inventive structures, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The active compound may be mixed under sterile conditions with a pharmaceutically-acceptable carrier, and with any preservatives, buffers, or propellants, which may be required.

The pastes, creams and gels may contain, in addition to the inventive structures, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. In some embodiments, the pastes, creams and gels may be formulated such that they may be used in a nebulizer or other device to facilitate transfer to the respiratory system. In some embodiments, the pastes, creams and gels may be administered by medical or surgical intervention directly to the tissue of the respiratory system (e.g., oral or nasal cavities, trachea, lungs, etc.).

Powders and sprays can contain, in addition to the structures described herein, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers, which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the inventive structures may be facilitated by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some embodiments, the HDL nanoparticles are administered to the subject by inhalation. In some embodiments, a suspension composition of the HDL nanoparticles deliverable from an inhaler such as a metered dose inhaler is provided. The suspension may include a suspension medium comprising a pharmaceutically acceptable HFA propellant; the synthetic HDL nanoparticles including any pharmaceutically acceptable variants thereof, suspended in the suspension medium. Several different inhaler systems are currently commercially available. Three common inhaler systems include dry powder inhalers, nebulizers and metered dose inhalers (MDIs). MDIs are active delivery devices that utilize the pressure generated by a propellant. Conventionally, chlorofluorocarbons (CFCs) have been used as propellants in MDI systems because of their low toxicity, desirable vapor pressure and suitability for formulation of stable suspensions.

Powder-based inhalers deliver the dose of powder using the energy generated by the patient's inspiratory effort and includes multi-use reservoir-based devices, re-usable devices supplied with unit-doses packaged in blisters, re-usable devices using unit-dose capsules loaded by the patient, and single-use disposable powder-based inhalers. Powder-based inhalers have been used mainly for maintenance treatment of respiratory diseases such as asthma or the chronic obstructive pulmonary disease. Single-use disposable powder devices for delivering the synthetic HDL nanostructures are particularly useful for treating an infectious agent.

In some embodiments, the HDS nanoparticles and/or compositions of the disclosure are administered to a subject systemically. Systemic administration can take place via enteral administration (e.g., absorption of the HDS nanoparticles and/or compositions of the disclosure through the gastrointestinal tract), absorption of the HDS nanoparticles and/or compositions of the disclosure through the respiratory system (e.g., inhalation, or any of the administration routes to the respiratory system disclosed herein), or parenteral administration (e.g., injection, infusion, or implantation).

Therapeutically Effective Amount

The term “therapeutically effective amount,” as may be used herein, refers to that amount of a material or composition comprising an inventive structure that is effective for producing some desired therapeutic effect in a subject at a reasonable benefit/risk ratio applicable to any medical treatment. Accordingly, a therapeutically effective amount may, for example, prevent, delay, minimize, or reverse disease progression associated with a disease or bodily condition. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

An effective amount may depend on the particular condition to be treated. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. In some cases, a maximum dose be used, that is, the highest safe dose according to sound medical judgment.

As used herein, the term “treating” or “treatment” refers to the application or administration of a synthetic nanostructure to a subject, who has a viral infection, a symptom of the viral infection, or a risk of exposure to the virus, with the purpose to prevent, cure, heal, alleviate, relieve, alter, remedy, ameliorate, improve, or affect the disorder resulting from the viral infection, the symptom of the infection, or the predisposition toward the infection.

In some instances the synthetic HDL nanostructure may be administered on demand. For instance, it may be administered to the subject when the subject has been exposed to a virus or respiratory virus or is at risk of being exposed to a virus or respiratory virus. In some embodiments, it may be administered to the subject when the subject has been infected by a virus or respiratory virus or is at risk of being infected by a virus or respiratory virus. In other instances it may be administered on a regular schedule such as once a day, twice a day, once every other day, once a week, twice a day, or once a day for one week to one month. In some embodiments, it may be administered once every other day, or some other increment (e.g., every second day, every third day, etc.). The synthetic HDL nanostructure may be mixed with or added to a food or drink product. For instance, it may be in a powder or liquid form that can be added to the food or drink. In some embodiments, it may be in the form of a spray, mist, inhalant, or other vehicle or formulation suitable for oral or intra-nasal administration. Alternatively it may be in a separate dosage form such as a capsule which can be delivered to the subject. The terms “administered” or delivered” are intended to encompass both administration by a health care worker as well as self-administration by a patient.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

A physician or veterinarian having ordinary skill in the art can readily determine and prescribe the effective amount of the pharmaceutical composition required. For example, the physician or veterinarian could start doses of the structures described herein employed in the pharmaceutical composition at levels lower than that required to achieve the desired therapeutic effect and then gradually increasing the dosage until the desired effect is achieved.

The present invention also provides any of the above-mentioned compositions useful for diagnosing, preventing, treating, or managing a disease or bodily condition packaged in kits, optionally including instructions for use of the composition. That is, the kit can include a description of use of the composition for participation in any disease or bodily condition, including those associated with abnormal lipid levels. The kits can further include a description of use of the compositions as discussed herein. The kit also can include instructions for use of a combination of two or more compositions described herein. Instructions also may be provided for administering the composition by any suitable technique, such as orally, intravenously, intra-nasally, or via another known route of drug delivery.

The kits described herein may also contain one or more containers, which can contain components such as the structures, signaling entities, and/or biomolecules as described. The kits also may contain instructions for mixing, diluting, and/or administrating the compounds. The kits also can include other containers with one or more solvents, surfactants, preservatives, and/or diluents (e.g., normal saline (0.9% NaCl), or 5% dextrose) as well as containers for mixing, diluting or administering the components to the sample or to the patient in need of such treatment.

The compositions of the kit may be provided as any suitable form, for example, as liquid solutions, mists, sprays, inhalants, or as dried powders. When the composition provided is a dry powder, the powder may be reconstituted by the addition of a suitable solvent, which may also be provided. In embodiments where liquid forms of the composition are used, the liquid form may be concentrated or ready to use. In embodiments where spray or mist forms of the composition are used, the spray or mist form may be concentrated or ready to use. In embodiments where spray or mist forms of the composition are used, the spray or mist form may be in a vial or container, or may come in packaging or a device for administration intra-nasally or for spray into the mouth or throat. The solvent will depend on the particular inventive structure and the mode of use or administration. Suitable solvents for compositions are well known and are available in the literature.

The kit, in one set of embodiments, may comprise one or more containers such as vials, tubes, and the like, each of the containers comprising one of the separate elements to be used in the method. For example, one of the containers may comprise a positive control in the assay. Additionally, the kit may include containers for other components, for example, buffers useful in the assay.

As used herein, a “subject” or a “patient” refers to any mammal (e.g., a human), for example, a mammal that may be susceptible to a disease or bodily condition such as the secondary diseases or conditions disclosed herein. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition. In some embodiments, a subject may be diagnosed with, or otherwise known to have, a disease or bodily condition associated with viral infection or respiratory viral infection, as described herein. In some embodiments, a subject may be diagnosed with, or otherwise known to have, a disease or bodily condition associated with abnormal lipid levels, as described herein. In certain embodiments, a subject may be selected for treatment on the basis of a known disease or bodily condition in the subject. In some embodiments, a subject may be selected for treatment on the basis of a suspected disease or bodily condition in the subject. In some embodiments, the composition may be administered to prevent the development of a disease or bodily condition. However, in some embodiments, the presence of an existing disease or bodily condition may be suspected, but not yet identified, and a composition of the invention may be administered to diagnose or prevent further development of the disease or bodily condition.

In some embodiments, the methods of the disclosure comprise administering any of the nanostructures of the disclosure and/or any of the pharmaceutical compositions of the disclosure to a subject's respiratory system. In some embodiments, the nanostructures of the disclosure and/or any of the pharmaceutical compositions of the disclosure are formulated into a liquid for administration. In some embodiments, the nanostructures of the disclosure and/or any of the pharmaceutical compositions of the disclosure are formulated into a spray for administration. In some embodiments, the nanostructures of the disclosure and/or any of the pharmaceutical compositions of the disclosure are formulated into a mist for administration. In some embodiments, the nanostructures of the disclosure and/or any of the pharmaceutical compositions of the disclosure are formulated into an inhalant for administration. In some embodiments, the nanostructures of the disclosure and/or any of the pharmaceutical compositions of the disclosure are formulated into an aerosol for administration. In some embodiments, the nanostructures of the disclosure and/or any of the pharmaceutical compositions of the disclosure are formulated into a powder for administration. In some embodiments, the methods of the disclosure comprise administering any of the nanostructures of the disclosure and/or any of the pharmaceutical compositions of the disclosure to a subject topically. In some embodiments, the topical administration is to a tissue. In some embodiments, the topical administration is topically to an internal tissue. In some embodiments, the methods of the disclosure comprise administering any of the nanostructures of the disclosure and/or any of the pharmaceutical compositions of the disclosure to a subject by oral administration. In some embodiments, the oral administration facilitates administration topically to an internal tissue. In some embodiments, the oral administration facilitates coating of the respiratory system of the subject with any of the nanostructures of the disclosure and/or any of the pharmaceutical compositions of the disclosure. In some embodiments, any of the nanostructures of the disclosure and/or any of the pharmaceutical compositions of the disclosure are formulated for topical administration. In some embodiments, any of the nanostructures of the disclosure and/or any of the pharmaceutical compositions of the disclosure are formulated for oral administration. In some embodiments, any of the nanostructures of the disclosure and/or any of the pharmaceutical compositions of the disclosure are formulated into a liquid. In some embodiments, the liquid is consumed orally. In some embodiments, the liquid is encapsulated. In some embodiments, the liquid is placed into a gel capsule for consumption. In some embodiments, the liquid is in a shell for consumption. In some embodiments, the liquid is in a pill for consumption. In some embodiments, the nanostructures of the disclosure and/or any of the pharmaceutical compositions of the disclosure are formulated into a powder. In some embodiments, the powder is consumed by the subject. In some embodiments, the powder is formulated into a pill. In some embodiments, the powder is mixable with a liquid. In some embodiments, the powder is encapsulated.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

Herein, a lipid nanoparticle drug was identified that, when administered to the respiratory system (e.g., orally, intra-nasally, via aerosol or inhalant, as a liquid, as a spray, as a mist) drastically inhibits viral entry into affected cells and prevents proliferation and infection by the virus. Accordingly, the lipid nanoparticle therapy has a tremendous number of applications regulating viral infection, proliferation, and subsequent virally caused diseases. The data herein show that cells exposed to virus in the presence of the HDL NPs of the instant disclosure, the number of virally infected cells is dramatically decreased (˜68%) as compared to those cells exposed to virus in the absence of the HDL NPs.

Nanostructure Synthesis

HDL NPs were synthesized using a 5 nm diameter gold nanoparticle (AuNP) core that is surface-functionalized with apoA-I and a phospholipid bilayer. The AuNP was chosen as the original core material because it is considered inert and nontoxic, monodisperse AuNPs can be readily synthesized, and it is a platform amenable to robust Au—S chemistry. The Au NPs were mixed with a 5-fold molar excess of purified human apolipoprotein (apoA-I). The AuNP/apoA-I mixture was incubated for 1 hr at room temperature (RT) on a flat bottom shaker at 60 rpm. Next, inner lipid 1,2-dipalmitoyl-sn-glycero-3-phosphoethanolamine-N-[3-(2-pyridyldithio)propionate] (PDP-PE) was dissolved in dichloromethane, chloroform, or ethanol (CH₂Cl₂, CHCl₃, or EtOH, at 1 mM) and was added to the AuNP/apoA-I solution in 250-fold molar excess to the Au NP.

In the synthesis of HDL NPs, the inner phospholipid contains a thiol or disulfide head-group in order to covalently attach the phospholipid to the gold nanoparticle. There are no restrictions on the identity of the outer leaflet phospholipid. The data show that the outer phospholipid layer can contain approximately 30% of the thiol or disulfide containing phospholipid under these synthesis conditions. The solution was vortexed, and then a 1:1 solution of cardiolipin (heart, bovine) (CL) and 1,2-dilinoleoyl-sn-glycero-3-phospho-(1′-rac-glycerol) (18:2 PG) were dissolved in CH₂Cl₂, CHCl₃, or EtOH, (1 mM) was added to the AuNP/apoA-I/PDP-PE solution at 250-fold molar excess to the AuNP and the solution was vortexed.

Several lipids and/or combinations of lipids can and have been used to generate the outer leaflet, including, but not limited to: 1,2-dipalmitoyl-sn-glycero-3-phosphocholine (DPPC), 1,2-dioleoyl-sn-glycero-3-phosphocholine (DOPC), 1,2-dipalmitoyl-sn-glycero-3-phosphoethanolamine (16:0), 1,2-distearoyl-sn-glycero-3-phosphoethanolamine (18:0 PE), and sphingomyelin. The solution was briefly vortexed and sonicated (˜2 minutes) until it became opaque and pink in color. The resulting mixture was gradually heated with constant stirring to evaporate the lipid solvent(s). Alternatively the mixture can be stirred overnight at RT to complete the reaction. The resultant HDL NPs were purified and concentrated using tangential flow filtration system. HDL NPs concentration was measured using UV-vis spectroscopy where Au NPs have a characteristic absorption at λₘₐₓ=520 nm, and the extinction coefficient 5 nm Au NPs is 9.696×10⁶ M⁻¹cm⁻¹.

### Example 1: VSV-G Pseudotyped Lentivirus

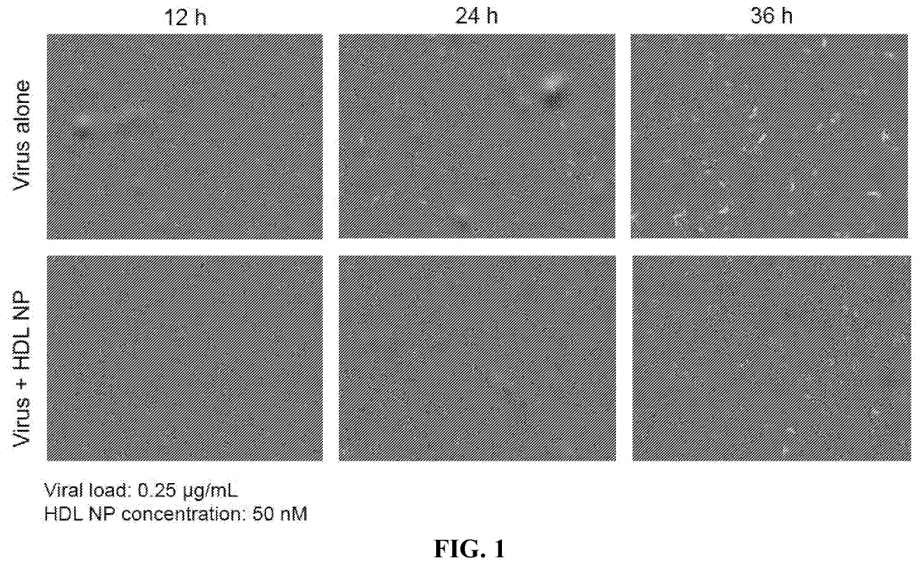

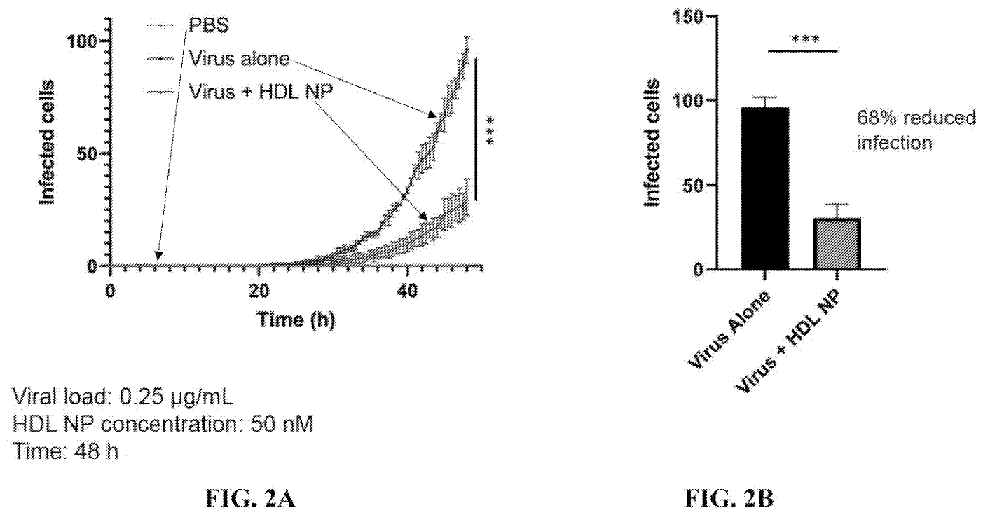

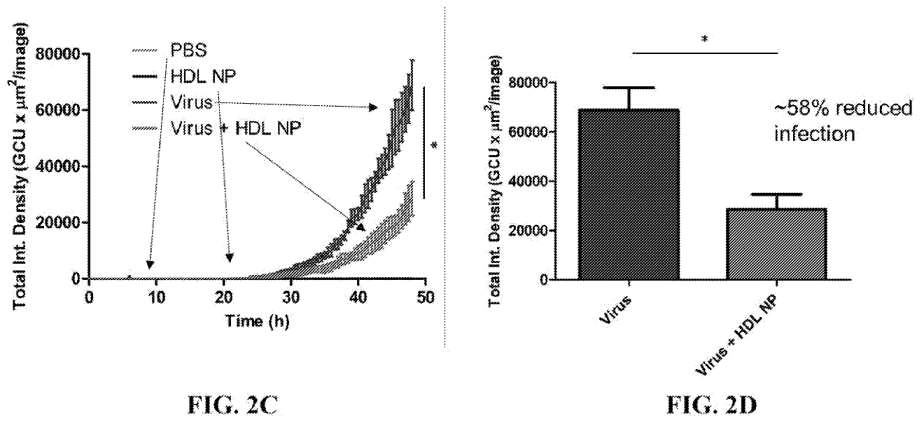

Human hepatoma (HepG2) cells were propagated in standard cell culture media and kept in a humidified incubation chamber. For experiments, HepG2 cells were sub-cultured into 24-well plates and seeded at 50,000 cells per well, 48 hours prior to infection. On the day of infection, HepG2 cells were approximately 60% confluent. VSV-G pseudotyped lentivirus encoding green fluorescence protein (GFP) was prepared in polybrene according to the manufacturer's instructions (EMD Millipore) and added to the HepG2 cells at either 0.25 μg/mL or 1 μg/mL to initiate infection. A solution of the HDL NPs (50 nM, final) was then added to the wells designated for treatment. All conditions were performed in triplicate. After treating cells with virus with or without HDL NP treatment, the 24-well plate was placed in an Incucyte S3 Live Cell Imaging System to monitor lentiviral infection via detection of GFP expression. Five images were taken in each well at 30 min intervals for a 48-hour period, using bright field and green fluorescence imaging. Images from 12 hours, 24 hours, and 36 hours are shown in FIG. 1. After 5 hours, the plate was briefly removed from the Incucyte to remove the media in each well, wash the cells, and replace with fresh media, in order to remove the lentivirus; the plate was then placed back in the Incucyte to continue live cell imaging. After incubation and imaging, the Incucyte S3 analysis software was used to quantify GFP positive cells. GFP positivity for each group consisted of the average of three wells, with values for each well-being the average of five distinct images (FIGS. 2A-2B). FIGS. 2C-2D provide the total integrated density of green fluorescence over time and at 48 hours. The data demonstrate that HDL NPs provide a statistically significant reduction in viral infection of HepG2 cells (˜58% reduced infection according to total density of green fluorescence), relative to untreated cells.

### Example 2: SARS-CoV-2-Pseudotype Lentivirus

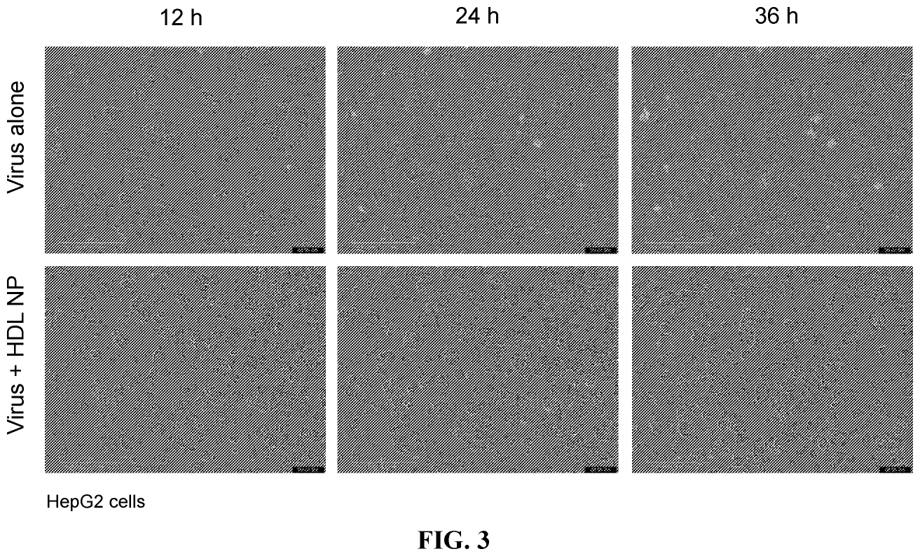

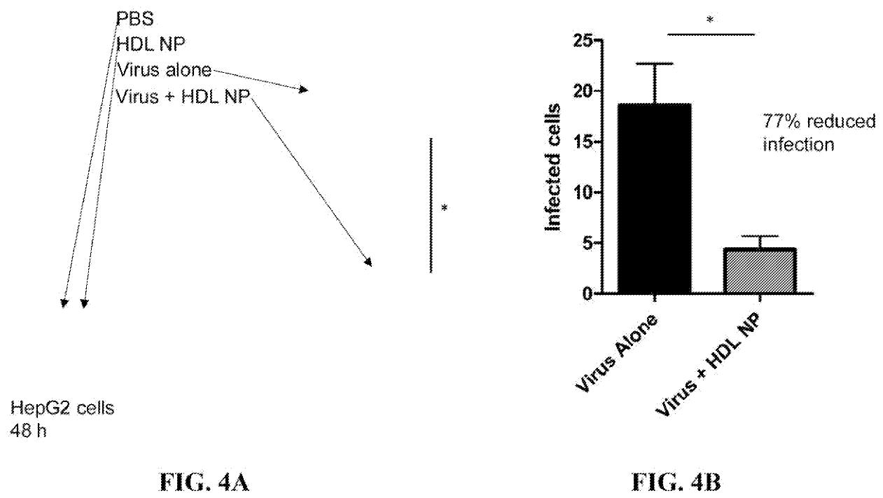

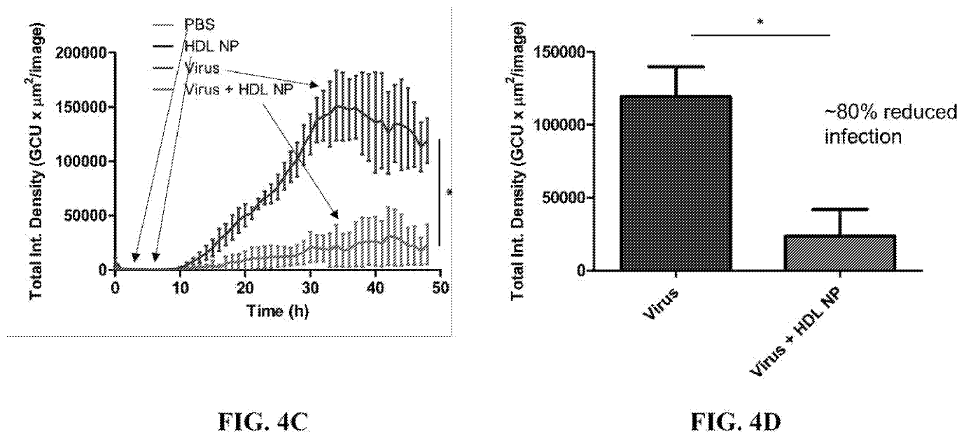

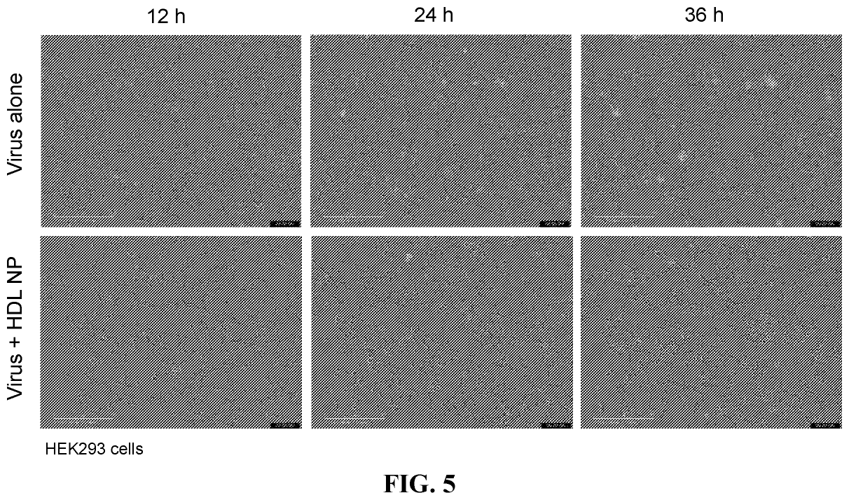

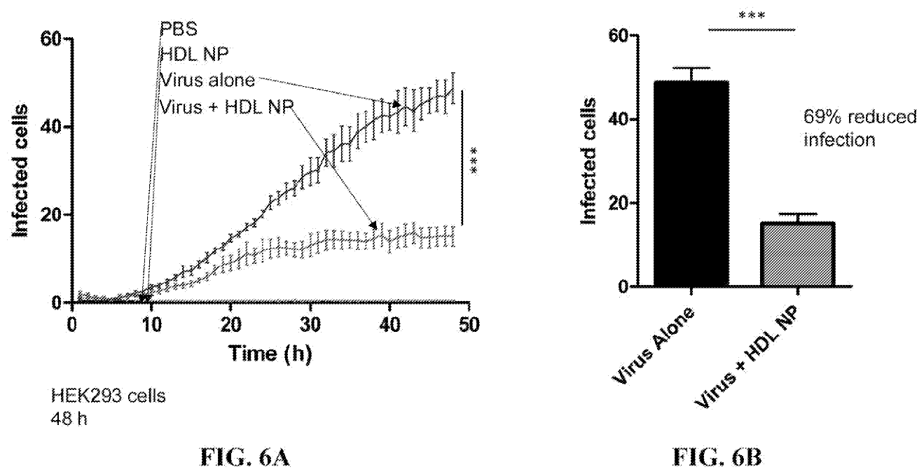

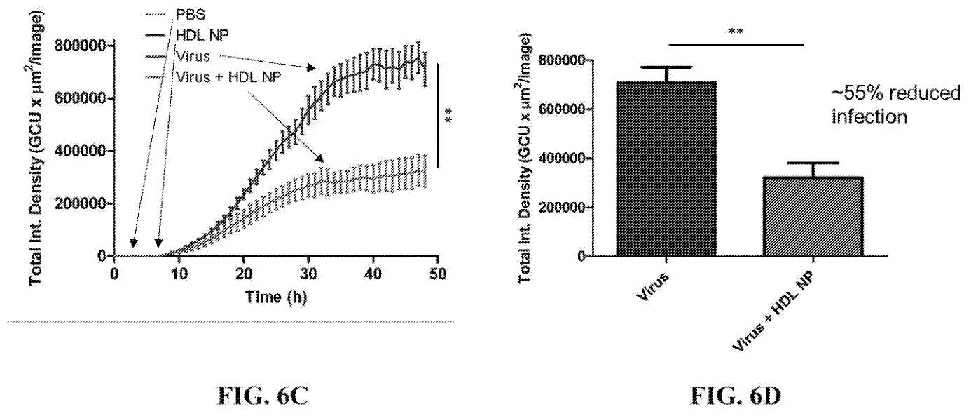

Human hepatoma (HepG2) and human embryonic kidney (HEK293) cells were propagated in standard cell culture media and kept in a humidified incubation chamber. For experiments, HepG2 cells were sub-cultured into 96-well plates and seeded at 10,000 cells per well, 24 hours prior to infection. SARS-CoV-2-pseudotype lentivirus encoding GFP was added to the HepG2 or HEK293 cells at 0.8 μL/100 μL to initiate infection. HDL NPs (50 nM, final) was then added to the media and 100 μL was applied to the wells for the pseudovirus+particle treatment (e.g., virus exposed with HDL NPs). For particle alone treatment, the HDL NPs were added (50 nM, final) to the media and 100 μL was added to each well. All conditions were performed in triplicate. After treating cells with virus with or without HDL NP treatment, the 96-well plate was placed in an Incucyte S3 Live Cell Imaging System to monitor lentiviral infection via detection of GFP expression. Four images were taken in each well at 1 hour intervals for a 48-hour period, using bright field and green fluorescence imaging. Images from 12 hours, 24 hours, and 36 hours are shown in FIG. 3 (HepG2) and FIG. 5 (HEK293). After incubation and imaging, the Incucyte S3 analysis software was used to quantify GFP positive cells. GFP positivity for each group consisted of the average of three wells, with values for each well-being the average of four distinct images (FIGS. 4A-4B, HepG2; FIGS. 6A-6B, HEK293). FIGS. 4C-4D and 6C-6D provide the total integrated density of green fluorescence over time and at 48 hours in the respective cell lines. The data demonstrate that HDL NPs provide a statistically significant reduction in infection of HepG2 cells (˜80% reduced infection according to total density of green fluorescence) and HEK293 cells (˜55% reduced infection according to total density of green fluorescence) with SARS-CoV-2 pseudovirus, relative to untreated cells.

### Example 3: Expression of SR-B1 in HEK293 and HepG2 Cells

HEK293 (ACE2) or HepG2 cells were plated at 400,000 cells per well in 6-well tissue culture plates. When cells had reached approximately 70% confluence, siRNA targeting SR-B1 and negative control scramble RNA were prepared using Lipofectamine RNAiMAX in Opti-MEM according to the manufacturer's instructions. Pre-prepared RNA (either siRNA targeting SR-B1, or negative control scramble RNA) was added to the cells at 30 nM and cells were incubated for 48 hours. Cell lysates were harvested using M-PER lysis buffer, samples were centrifuged for 10 min at 14,000×g to pellet cellular debris. The supernatant was then transferred to a new tube and protease and phosphatase inhibitors were added prior to processing for western blot.

Western blotting was then performed. Total sample protein concentration was determined using bicinchoninic acid (BCA) assay. Samples were then normalized to total protein, mixed with 4×Laemmli loading buffer containing β-mercaptoethanol, and boiled for 10 minutes at 100° C. Proteins were resolved using a 4%-20% polyacrylamide gel (120 V, 1 h) and transferred to a 0.45 μm PVDF membrane (60 V, 1 h). The membrane was blocked using 5% milk in Tris buffered saline (TBS) and Tween-20 (0.1%) for 1 hour. SR-B1 antibody was applied (1:2000) (Abcam, ab52629) and incubated overnight at 4° C. Blot was washed 10 minutes (3×) in TBST (0.1% Tween-20) secondary goat anti-rabbit antibody (BioRad, 1721019) was applied (1:1000) for 1 hour at R.T. and blot was washed (3×) same as described above. Protein was detected using enhanced chemiluminescence (ECL) detection (Bio-Rad, 1705060) and an Azure 300 (Azure Biosystems) gel imaging system.

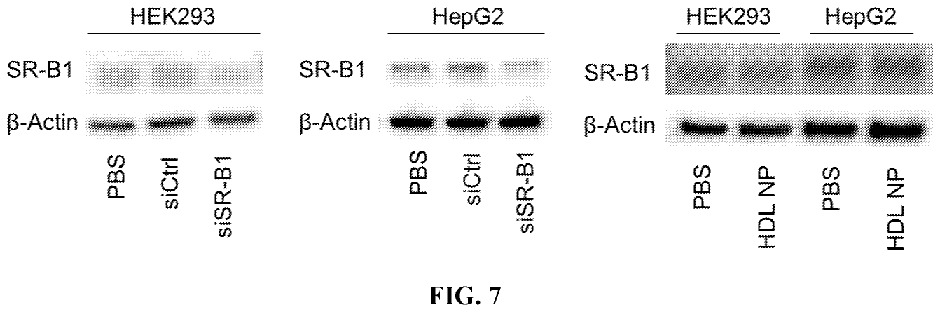

As shown in FIG. 7, expression of scavenger receptor type B-1 (SR-B1) was inhibited by the siRNA that targets SR-B1 (siSR-B1) relative to scramble RNA control in both HEK293 and HepG2 cells. These data demonstrate that HEK293 and HepG2 cells express SR-B1 and are thus potential targets for agents that target SR-B1 (such as siRNA or HDL NPs).

### Example 4: Knockdown of SR-B1 Attenuates HDL NP Inhibition

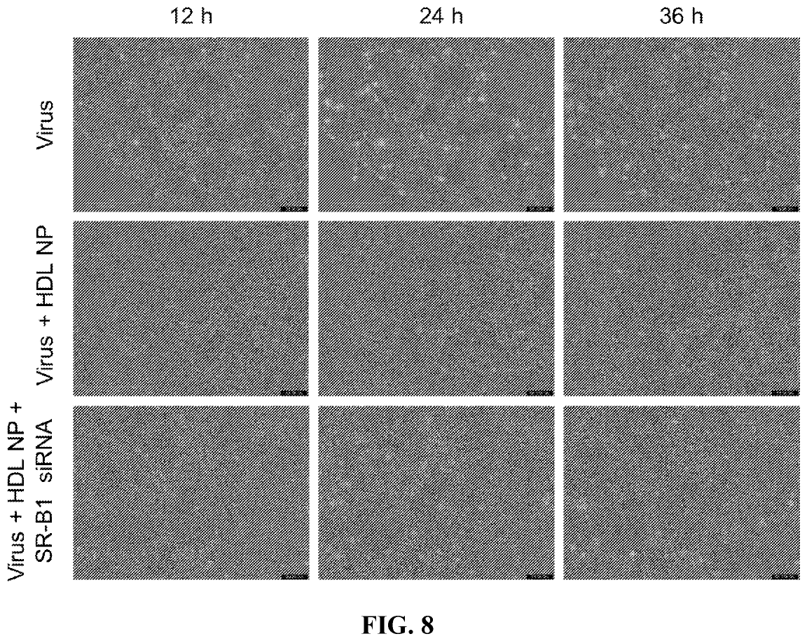

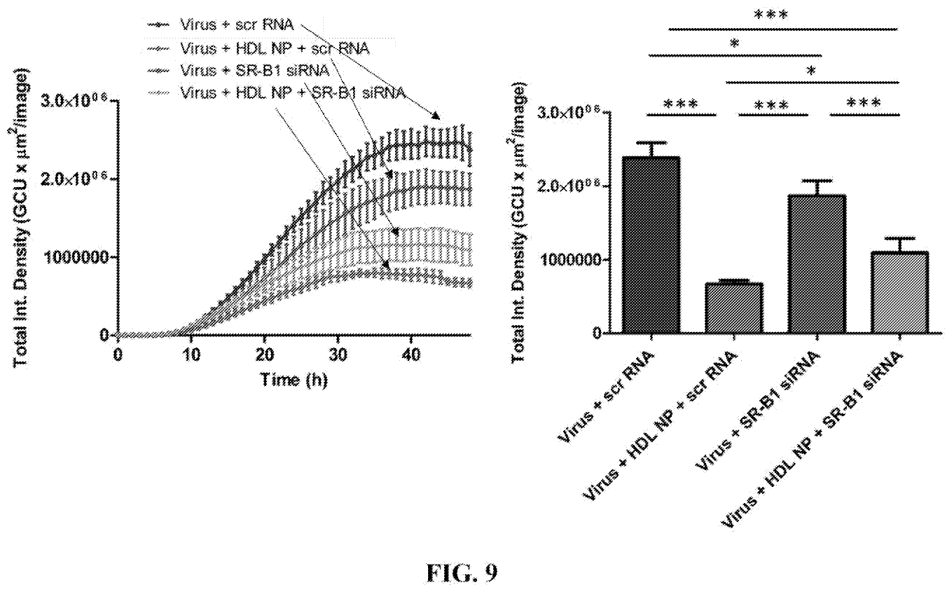

Human embryonic kidney (HEK293) cells were propagated in standard cell culture media and kept in a humidified incubation chamber. For experiments, HEK293 cells were sub-cultured into 96-well plates and seeded at 10,000 cells per well, 24 hours prior to infection. SARS-CoV-2-pseudotype lentivirus encoding GFP was added to the HEK293 cells at 0.8 μL/100 μL to initiate infection. HDL NPs (50 nM) and scramble RNA control (60 nM), or HDL NPs (50 nM) and SR-B1 siRNA (60 nM) were then added to infected cells. All conditions were performed in triplicate. After treatment, the 96-well plates were placed in an Incucyte S3 Live Cell Imaging System to monitor lentiviral infection via detection of GFP expression. Four images were taken in each well at 1 hour intervals for a 48-hour period, using bright field and green fluorescence imaging. Images from 12 hours, 24 hours, and 36 hours are shown in FIG. 8. After incubation and imaging, the Incucyte S3 analysis software was used to quantify total integrated density of green fluorescence, a quantitative representation of viral infectivity. Total integrated density of green fluorescence for each group consisted of the average of three wells, with values for each well-being the average of four distinct images (FIG. 9).

Treatment of cells with HDL NPs and scramble RNA provided a statistically significant reduction in viral infection of the cells, relative to control with scramble RNA only. Treatment of cells with HDL NPs and scramble RNA also provided a reduction in viral infection relative to cells treated with HDL NPs and SR-B1 siRNA. These data demonstrate that the anti-viral mechanism of HDL NPs is mediated in part by SR-B1, because knocking down SR-B1 attenuated the HDL NP inhibition of viral infection.

### Example 5: Inhibition of SR-B1 Leads to Inhibition of SARS-CoV-2 Infection

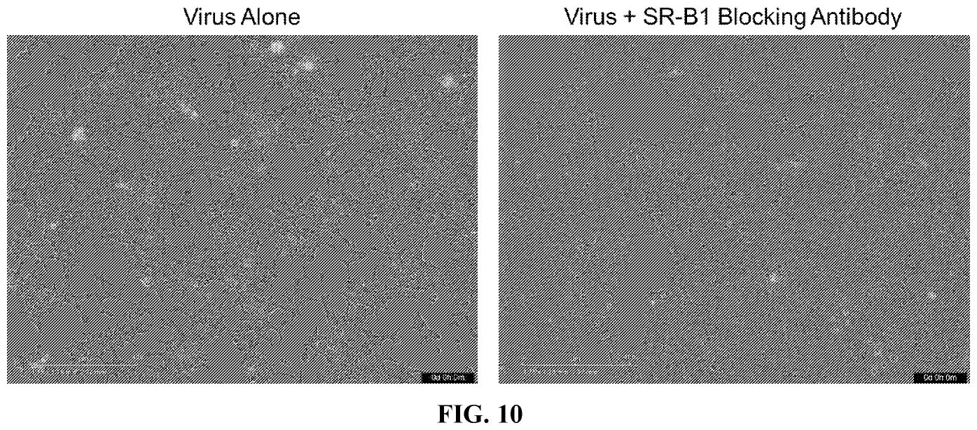

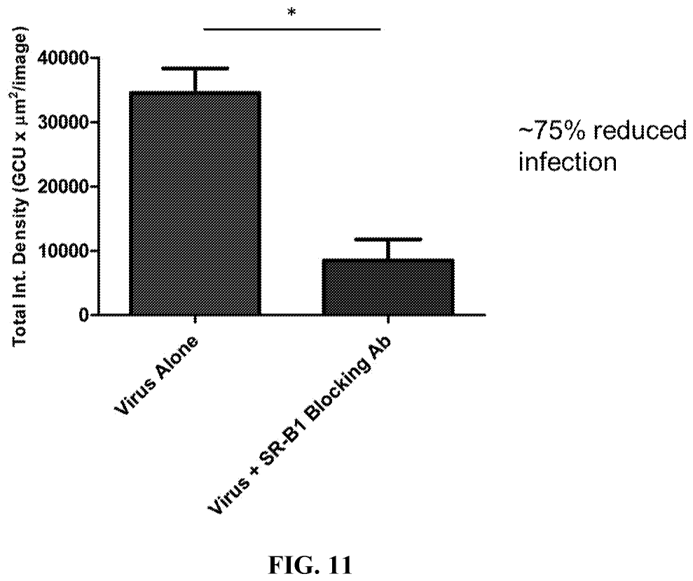

Human embryonic kidney (HEK293) cells were propagated in standard cell culture media and kept in a humidified incubation chamber. For experiments, HEK293 cells were sub-cultured into 96-well plates and seeded at 10,000 cells per well, 24 hours prior to infection. SARS-CoV-2-pseudotype lentivirus encoding GFP was added to the HEK293 cells at 0.8 μL/100 μL to initiate infection. Infected cells were either treated with saline (control) or an SR-B1 blocking antibody (20 μg/mL) (an antibody that specifically binds to SR-B1 protein). All conditions were performed in triplicate. After treatment, the 96-well plates were placed in an Incucyte S3 Live Cell Imaging System to monitor lentiviral infection via detection of GFP expression. Four images were taken in each well at 1 hour intervals for a 48-hour period, using bright field and green fluorescence imaging. Images from the 48-hour time point is shown in FIG. 10. After incubation and imaging, the Incucyte S3 analysis software was used to quantify total integrated density of green fluorescence, a quantitative representation of viral infectivity. Total integrated density of green fluorescence for each group consisted of the average of three wells, with values for each well-being the average of four distinct images (FIG. 11).

Cells treated with the SR-B1 blocking antibody had a 75% reduction in viral infection relative to control (cells treated with saline). These data demonstrate that SR-B1 is influential in the mechanism of viral infection (e.g., SARS-CoV-2 infection) and show that that inhibition of SR-B1 is a potent mechanism for treating viral infection (e.g., SARS-CoV-2 infection).

### REFERENCES

1. Galanti, M. et al., Rates of asymptomatic respiratory virus infection across age groups, Epidemiol Infect. 2019; 147: e176.

### Other Embodiments

Embodiment 1. A method for treating a viral infection in a subject, comprising administering to a subject having a viral infection, a synthetic HDL nanostructure in an effective amount to inhibit viral entry into cells of the subject in order to treat the viral infection.

Embodiment 2. The method of embodiment 1, wherein the synthetic HDL nanoparticle is delivered to the subject's respiratory system.

Embodiment 3. The method of embodiment 1, wherein the subject is identified as having a respiratory viral infection caused by a respiratory virus.

Embodiment 4. The method of embodiment 3, wherein the respiratory virus is selected from the group consisting of: adenovirus (ADV); influenza virus, human bocavirus (HBoV); human coronavirus (HCoV); human metapneumovirus (HMPV); human parainfluenza virus (HPIV); human respiratory syncytial virus (HRSV); human rhinovirus (HRV); severe acute respiratory syndrome coronavirus (SARS-CoV); and Middle East Respiratory Syndrome coronavirus (MERS-CoV).

Embodiment 5. The method of embodiment 3, wherein the virus is a coronavirus.

Embodiment 6. The method of embodiment 5, wherein the coronavirus is a SARS-CoV or a MERS-CoV.

Embodiment 7. The method of embodiment 3, wherein the virus is a respiratory syncytial virus.

Embodiment 8. The method of embodiment 1, wherein the subject is identified as having a viral infection with a virus that infects a scavenger receptor type B-1 (SR-B1) and/or CD-36 receptor positive cell.

Embodiment 9. A method for treating severe acute respiratory syndrome coronavirus 2 (SARS-CoV-2) infection in a subject, comprising administering to a subject infected with SARS-CoV-2 a synthetic HDL nanostructure in an effective amount to treat the SARS-CoV-2 infection in the subject.

Embodiment 10. The method of any one of embodiments 1-9, wherein the synthetic HDL nanostructure comprises a nanostructure core; an apolipoprotein; and a shell comprising a lipid surrounding and attached to the nanostructure core, wherein the shell comprises a phospholipid.

Embodiment 11. The method of embodiment 10, wherein the apolipoprotein is apolipoprotein A-I, apolipoprotein A-II, or apolipoprotein E.

Embodiment 12. The method of embodiment 10, wherein the nanostructure further comprises a cholesterol.

Embodiment 13. The method of embodiment 10, wherein the shell substantially surrounds the nanostructure core.

Embodiment 14. The method of embodiment 10, wherein the shell comprises a lipid monolayer.

Embodiment 15. The method of embodiment 10, wherein the shell comprises a lipid bilayer.

Embodiment 16. The method of embodiment 15, wherein at least a portion of the lipid bilayer is covalently bound to the core.

Embodiment 17. The method of embodiment 10, wherein the core of the synthetic HDL nanostructure has a largest cross-sectional dimension of less than or equal to about 5 nanometers (nm).

Embodiment 18. The method of embodiment 10, wherein the nanostructure core is an inorganic nanostructure core.

Embodiment 19. The method of embodiment 10, wherein the nanostructure core comprises gold.

Embodiment 20. The method of embodiment 10, wherein the nanostructure core is an organic nanostructure core.

Embodiment 21. The method of embodiment 10, wherein the synthetic HDL nanostructure has a diameter of less than or equal to about 15 nanometers (nm).

Embodiment 22. The method of any one of the preceding embodiments, wherein the synthetic HDL nanostructure is administered to the subject once or twice a day.

Embodiment 23. The method of any one of embodiments 1-21, wherein the synthetic HDL nanostructure is administered to the subject once every other day.

Embodiment 24. The method of any one of embodiments 1-21, wherein the synthetic HDL nanostructure is administered to the subject in combination with an anti-inflammatory agent.

Embodiment 25. The method of any one of embodiments 1-21, wherein the synthetic HDL nanostructure is administered to the subject by inhalation.

Embodiment 26. The method of any one of embodiments 1-21, wherein the synthetic HDL nanostructure is administered to the subject systemically.

Embodiment 27. The method of any one of embodiments 1-26, wherein the synthetic HDL nanostructure is administered to the subject by intranasal administration.

Embodiment 28. The method of any one of embodiments 1-26, wherein the synthetic HDL nanostructure is administered to the subject by oral administration with an oral dosage form that is a liquid, a spray or mist.

Embodiment 29. The method of any one of embodiments 1-26, wherein the synthetic HDL nanostructure is administered to the subject by intranasal administration.

Embodiment 30. The method of any one of embodiments 1-29, wherein the subject is identified as having a comorbid disorder selected from the group consisting of hypertension, cardiovascular disease, obesity, and diabetes.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating a viral infection in a subject, comprising administering to a subject having a viral infection, an agent that targets cholesterol metabolism in an effective amount to inhibit viral entry into cells of the subject in order to treat the viral infection.

2. The method of claim 1, wherein the agent that targets cholesterol metabolism is delivered to the subject's respiratory system.

3. The method of claim 1 or 2, wherein the subject is identified as having a respiratory viral infection caused by a respiratory virus.

4. The method of claim 3, wherein the respiratory virus is selected from the group consisting of: adenovirus (ADV); influenza virus, human bocavirus (HBoV); human coronavirus (HCoV); human metapneumovirus (HMPV); human parainfluenza virus (HPIV); human respiratory syncytial virus (HRSV); human rhinovirus (HRV); severe acute respiratory syndrome coronavirus (SARS-CoV); and Middle East Respiratory Syndrome coronavirus (MERS-CoV).

5. The method of claim 3, wherein the virus is a coronavirus.

6. The method of claim 5, wherein the coronavirus is a SARS-CoV or a MERS-CoV.

7. The method of claim 3, wherein the virus is a respiratory syncytial virus.

8. The method of any one of claims 1-7, wherein the subject is identified as having a viral infection with a virus that infects a scavenger receptor type B-1 (SR-B1), CD-36, LDL-R, or ACE2 receptor positive cell.

9. A method for treating severe acute respiratory syndrome coronavirus 2 (SARS-CoV-2) infection in a subject, comprising administering to a subject infected with SARS-CoV-2 an agent that targets cholesterol metabolism in an effective amount to treat the SARS-CoV-2 infection in the subject.

10. The method of any one of claims 1-9, wherein the agent that targets cholesterol metabolism is a synthetic HDL nanostructure, an inhibitory nucleic acid that targets a cholesterol metabolism gene, or an antibody that inhibits the function of a protein encoded by a cholesterol metabolism gene.

11. The method of claim 10, wherein the cholesterol metabolism gene is scavenger receptor type B-1 (SR-B1), CD36, low-density lipoprotein receptor (LDL-R), or Angiotensin-Converting Enzyme 2 (ACE2).

12. The method of any one of claims 1-11, wherein the agent that targets cholesterol metabolism inhibits the function of a cell-surface receptor, optionally wherein the cell-surface receptor is scavenger receptor type B-1 (SR-B1), CD36, low-density lipoprotein receptor (LDL-R), or Angiotensin-Converting Enzyme 2 (ACE2).

13. The method of any one of claims 10-12, wherein the synthetic HDL nanostructure comprises a nanostructure core; an apolipoprotein; and a shell comprising a lipid surrounding and attached to the nanostructure core, wherein the shell comprises a phospholipid.

14. The method of claim 13, wherein the apolipoprotein is apolipoprotein A-I, apolipoprotein A-II, or apolipoprotein E.

15. The method of claim 13 or 14, wherein the nanostructure further comprises a cholesterol.

16. The method of any one of claims 13-15, wherein the shell substantially surrounds the nanostructure core.

17. The method of any one of claims 13-16, wherein the shell comprises a lipid monolayer.

18. The method of any one of claims 13-16, wherein the shell comprises a lipid bilayer.

19. The method of claim 18, wherein at least a portion of the lipid bilayer is covalently bound to the core.

20. The method of any one of claims 13-19, wherein the core of the synthetic HDL nanostructure has a largest cross-sectional dimension of less than or equal to about 5 nanometers (nm).

21. The method of any one of claims 13-20, wherein the nanostructure core is an inorganic nanostructure core.

22. The method of any one of claims 13-20, wherein the nanostructure core comprises gold.

23. The method of any one of claims 13-20, wherein the nanostructure core is an organic nanostructure core.

24. The method of any one of claims 13-23, wherein the synthetic HDL nanostructure has a diameter of less than or equal to about 15 nanometers (nm).

25. The method of claim 10, wherein the inhibitory nucleic acid that targets a cholesterol metabolism gene is an siRNA that targets SR-B1.

26. The method of claim 10, wherein the antibody that inhibits the function of a protein encoded by a cholesterol metabolism gene is an anti-SR-B1 antibody (i.e., an antibody that specifically binds to SR-B1.

27. The method of any one of the preceding claims, wherein the agent that targets cholesterol metabolism is administered to the subject once or twice a day.

28. The method of any one of claims 1-26, wherein the agent that targets cholesterol metabolism is administered to the subject once every other day.

29. The method of any one of claims 1-28, wherein the agent that targets cholesterol metabolism is administered to the subject in combination with an anti-inflammatory agent.

30. The method of any one of claims 1-29, wherein the agent that targets cholesterol metabolism is administered to the subject by inhalation.

31. The method of any one of claims 1-29, wherein the agent that targets cholesterol metabolism is administered to the subject systemically.

32. The method of any one of claims 1-29, wherein the agent that targets cholesterol metabolism is administered to the subject by intranasal administration.

33. The method of any one of claims 1-29, wherein the agent that targets cholesterol metabolism is administered to the subject by oral administration with an oral dosage form that is a liquid, a spray or mist.

34. The method of any one of claims 1-29, wherein the agent that targets cholesterol metabolism is administered to the subject by intranasal administration.

35. The method of any one of claims 1-34, wherein the subject is identified as having a comorbid disorder selected from the group consisting of hypertension, cardiovascular disease, obesity, and diabetes.
